SEQUENCE LISTING (55 sequences)

SEQ ID NO: 1 - protein, 77 aa, Homo sapiens [sequence omitted]

SEQ ID NO: 2 - protein, 88 aa, Homo sapiens
  misc_feature: Incyte ID No 1329044CD1
MKTPNDLFLRQLGYLSICCFVFSSEESKNYKISLIVYLTFLTMETKPRNSIYSVLTQSTHPDFESPRTGVPNPRAEDQYQFEAYYRVT

SEQ ID NO: 3 - protein, 96 aa, Homo sapiens
  misc_feature: Incyte ID No 1493630CD1
MSMQFLFKMVALCCCLWKISGCEEVPLTYNLLKCLLDKAHCVLLTPCGYIFSLISPEILKLTLITLQILLILKNLHLLWLTVSSRCVHRSSARKEK

SEQ ID NO: 4 - protein, 104 aa, Homo sapiens
  misc_feature: Incyte ID No 1533041CD1
MRLSLPLGSLLWPFLVCGCLLQVALCQTRSAPHLDTHSPVAFQCSGRKPVSLDVKLTLMGWGRGLGRRGGRGEGTELRISWSALQAQRRSAKVLNRFSLEIKNP

SEQ ID NO: 5 - protein, 60 aa, Homo sapiens
  misc_feature: Incyte ID No 1566162CD1
MLMFIKGLSSTLFLGSTLSHRDPICFYSFHFHLYLLPHAVSPVTNSIYNYLLGLYLDTCT

SEQ ID NO: 6 - protein, 117 aa, Homo sapiens
  misc_feature: Incyte ID No 1811831CD1
MPKSQSHHLTQLQLLPSCLLGLLPPVPAVHAYILQGCVLSGREIFFSVLQFFTQTFSFVVPVFPSFPGGFRLPFSSPWLSLCPIHRSTLQACLYERGLFLCRKLTLTRCGCSYTDLI

SEQ ID NO: 7 - protein, 86 aa, Homo sapiens [sequence omitted]

SEQ ID NO: 8 - protein, 109 aa, Homo sapiens [sequence omitted]

SEQ ID NO: 9 - protein, 111 aa, Homo sapiens
  misc_feature: Incyte ID No 4318494CD1
MRSPSFPFTLLSGLPGPGFSQLCVRVSQVSRNPMRSEGCFGLLKSVQDNPASALELLDFSDIQVNAEFDGLASSVRGILPELCIKTGACRVEYKKELLPVFRSALPASVPK

SEQ ID NO: 10 - protein, 182 aa, Homo sapiens
  misc_feature: Incyte ID No 5090841CD1
MEPQLGPEAAALRPGWLALLLWVSALSCSFSLPASSLSSLVPQVRTSYNFGRTFLGLDKCNACIGTSICKKFFKEEIRSDNWLASHLGLPPDSLLSYPANYSDDSKIWRPVEIFRLVSKYQNEISDRRICASASAPKTCSIERVLRKTERFQKWLQAKRLTPDLVQDCHQGQRELKFLCMLR

SEQ ID NO: 11 - protein, 105 aa, Homo sapiens
  misc_feature: Incyte ID No 2006548CD1
MRGATRVSIMLLLVTVSDCAVITGACERDVQCGAGTCCAISLWLRGLRMCTPLGREGEECHPGSHKVPFFRKRKHHTCPCLPNLLCSRFPDGRYRCSMDLKNINF

SEQ ID NO: 12 - protein, 342 aa, Homo sapiens [sequence omitted]

SEQ ID NO: 13 - protein, 451 aa, Homo sapiens
  misc_feature: Incyte ID No 2267403CD1
MVPEVRVLSSLLGLALLWFPLDSHARARPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHKKACVHGGKTYSHGEVWHPAFRAFGPLPCILCTCEDGRQDCQRVTCPTEYPCRHPEKVAGKCCKICPEDKADPGHSEISSTRCPKAPGRVLVHTSVSPSPDNLRRFALEHEASDLVEIYLWKLVKDEETEAQRGEVPGPRPHSQNLPLDSDQESQEARLPERGTALPTARWPPRRSLERLPSPDPGAEGHGQSRQSDQDITKT

SEQ ID NO: 14 - protein, 189 aa, Homo sapiens
  misc_feature: Incyte ID No 2933038CD1
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 15 - protein, 216 aa, Homo sapiens
  misc_feature: Incyte ID No 3216587CD1
MGAVMGTFSSLQTKQRRPSKDKIEDELEMTMVCHRPEGLEQLEAQTNFTKRELQVLYRGFKNECPSGVVNEDTFKQIYAQFFPHGDASTYAHYLFNAFDTTQTGSVKFEDFVTALSILLRGTVHEKLRWTFNLYDINKDGYINKEEMMDIVKAIYDMMGKYTYPVLKEDTPRQHVDVFFQKMDKNKDGIVTLDEFLESCQEDDNIMRSLQLFQNVM

SEQ ID NO: 16 - protein, 178 aa, Homo sapiens
  misc_feature: Incyte ID No 5037143CD1
MAAARLCLSLLLLSTCVALLLQPLLGAQGAPLEPVYPGDNATPEQMAQYAADLRRYINMLTRPRCVPQLGREIPAPGTLGPLHIPGHTLSPAPAPAPSRPALGKTGHLCSTGLDQCALGKMVPTGRYETGGLAPGHSACPCCLFPPRYGKRHKEDTLAFSEWGSPHAAVPRELSPLDL

SEQ ID NO: 17 - protein, 177 aa, Homo sapiens [sequence omitted]

SEQ ID NO: 18 - protein, 179 aa, Homo sapiens
  misc_feature: Incyte ID No 5571181CD1
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 19 - protein, 213 aa, Homo sapiens
  misc_feature: Incyte ID No 685374CD1
MALLRKSYSEPQLKGIVTKLYSRQGYHLQLQADGTIDGTKDEDSTYTLFNLIPVGLRVVAIQGVQTKLYLAMNSEGYLYTSELFTPECKFKESVFENYYVTYSSMIYRQQQSGRGWYLGLNKEGEIMKGNHVKKNKPAAHFLPKPLKVAMYKEPSLHDLTEFSRSGSGTPTKSRSVSGVLNGGKSMSHNESTPVRAKEGLCNRTLPPGAVEFF

SEQ ID NO: 20 - protein, 239 aa, Homo sapiens
  misc_feature: Incyte ID No 843193CD1
MAICPLHSAGQVACPHYIHLLTPLPWMDQWWCHPKQIDTIFPLVTAKGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPLTNHAEKQKQFEFVGSAPTRRAKRTRRPQPLT

SEQ ID NO: 21 - protein, 493 aa, Homo sapiens
  misc_feature: Incyte ID No 1359783CD1
MLKALFLTMLTLALVKSQDTEETITYTQCTDGYEWDPVRQQCKDIDECDIVPDACKGGMKCVNHYGGYLCLPKTAQIIVNNEQPQQETQPAEGTSGATTGVVAASSMATSGVLPGGGFVASAAAVAGPEMQTGRNNFVIRRNPADPQRIPSNPSHRIQCAAGYEQSEHNVCQDIDECTAGTHNCRADQVCINLRGSFACQCPPGYQKRGEQCVDIDECTIPPYCHQRCVNTPGSFYCQCSPGFQLAANNYTCVDINECDASNQCAQQCYNILGSFICQCNQGYELSSDRLNCEDIDECRTSSYLCQYQCVNEPGKFSCMCPQGYQVVRSRTCQDINECETTNECREDEMCWNYHGGFRCYPRNPCQDPYILTPENRCVCPVSNAMCRELPQSIVYKYMSIRSDRSVPSDIFQIQATTIYANTINTFRIKSGNENGEFYLRQTSPVSAMLVLVKSLSGPREHIVDLEMLTVSSIGTFRTSSVLRLTIIVGPFSF

SEQ ID NO: 22 - protein, 121 aa, Homo sapiens
  misc_feature: Incyte ID No 1440015CD1
MARRAGGARMFGSLLLFALLAAGVAPLSWDLPEPRSRASKIRVHSRGNLWATGHFMGKKSLEPSSPSPLGTAPHTSLRDQRLQLSHDLLGILLLKKALGVSSAAPHPKSSTGGCWYKYLQK

SEQ ID NO: 23 - protein, 116 aa, Homo sapiens
  misc_feature: Incyte ID No 1652885CD1
MVPQPPTTCPWKPVPSPCDLRVQGICPSSFPDTPLAQEEDSEPLPPQDAQTSGSLLHYLLQAMERPGRSQAFLFQPQRFGRNTQGSWRNEWLSPRAGEGLNSQFWSLAAPQRFGKK

SEQ ID NO: 24 - protein, 136 aa, Homo sapiens
  misc_feature: Incyte ID No 4003984CD1
MQRWTLWAAAFLTLHSAQAFPQTDISISPALPELPLPSLCPLFWMEFKGHCYRFFPLNKTWAEADLYCSEFSVGRKSAKLASIHSWEENVFVYDLVNSCVPGIPADVWTGLHDHRQVRKQWPLGPLGSSSQDSILI

SEQ ID NO: 25 - protein, 176 aa, Homo sapiens
  misc_feature: Incyte ID No 4365383CD1
MNFVHTSRKVKSLNPKKFSIHDQDHKVLVLDSGNLIAVPDKNYIRPEIFFALASSLSSASAEKGSPILLGVSKGEFCLYCDKDKGQSHPSLQLKKEKLMKLAAQKESARRPFIFYRAQVGSWNMLESAAHPGWFICTSCNCNEPVGVTDKFENRKHIEFSFQPVCKAEMSPSEVSD

SEQ ID NO: 26 - protein, 134 aa, Homo sapiens
  misc_feature: Incyte ID No 5497814CD1
MSVLPLCVLPLLLASCSHLSTFLWPPSLACCLETLVGIPFSRHRSLGLIPAPRCLPLPAAIPTSLCSPPFHSLHSLPRCPLLKVLGHPQVAWSRQQPLHFTSANDRHLSKACPGCSWYSSDSLVAFQRPFPSGL

SEQ ID NO: 27 - DNA, 2730 bp, Homo sapiens
  misc_feature: Incyte ID No 1288847CB1
cgggtaggaagctcctcttagtactaagagacttcaagcttcttgctttaagtcctcaccctttacattatctaattcttcagttttgatgctgatacctgcccccggccctaccttagctctgtggcattatatctcctctctgggactcttcaacctggtactccatacctcttgtgccctctcactttaggcagcttgcactattcttgaatgaatgaagaattatttcctcatttggaagtaggagggactgaagaaattctccccaggcactgtgggactgagagtcctattcccctagtaataggtcatattcccctagtaatatgagttctcaaagcctacattcaggatctccctctaggatgtgatagatctggtccctctccttgaactacccctccacacgctctagtcccttcaacctaccggtctattaagtggtggcttttctctccttggagtgccccaattttatattctcaggggccaaggctaggtctgcaaccctctgtctctgacagattgggagccacaggtgcctaattgggaaccagggcatgggaaaggagtgggtcaaaattcttctctttctcctccacctctcaaacttcttcactatagtgaccttcctaggctctcaggggctccttcagtccccatcctatgagaaactagtgggttgctgcctgatgacaaggggttgtttcagcccctcagtcatgctgccttctgctgctccctcccagcaggattcaccctctcattcccgggctcctgggccctgttcttaggatcagtggcagggagaaacgggtatctcttttctctcttctaattttcagtataaccaaaaattatcccagcatgagcacgggcacgtgcccttcaccccattccacccttgttccagcaagactgggatgggtacaactgaactggggtcttcctttactacccccttctacactcagctcccagacacagggtaggaggggggactgctggctactgcagagacccttggctatttgagtaacctaggattagtgagaaggggcagaaggagatacaactccactgcaagtggaggtttctttctacaagagttttctgcccaaggccacagccatcccactctctgcttccttgagattcaaaccaaaggctgtttttctatgtttaaagaaaaaaaaaagtaaaaaccaaacacaacacctcacaagttgtaactcttggtccttctctctctccttttctcttcccttccttccccttccatctttctttccacatgtcctttccttattggctcttttacctcctacttttctcactccctatcagggatattttggggggggatggtaaagggtgggctaaggaacagaccctgggattagggccttaagggctctgagaggagtctaccttgccttcttatgggaagggagaccctaaaaaactttctcctctttgtcctcctttttctcccccactctgaggtttccccaagagaaccagattggcagggagaagcattgtggggcaattgttcctccttgacaatgtagcaataaatagatgctgccaagggcagaaaatggggaggttagctcagagcagagtagtctctagagaaaggaagaatcctcaacggcaccctggggtgctagctcctttttagaatgtcagcagagctgagattaatatctgggcttttcctgaactattctggttattgagcccttcctgttagacctaccgcctcccacctcttctgtgtctgctgtgtatttggtgacacttcataaggactagtcccttctggggtatcagagccttagggtgcccccatccccttccccagtcaactgtggcacctgtaacctcccggaacatgaaggactatgctctgaggctatactctgtgcccatgagagcagagactggaagggcaagaccaggtgctaaggaggggagagggggcatcctgtctctctccagaccatcactgcactttaaccagggtcttaggtacaaaatcctacttttcagagccttccagctctggaacctcaaacatcctcatgctctctcccagctccttttgcataaaaaaaaaagtaaagaaaaagaaaaaaaaatacacacacactgaaacccacatggagaaaagaggtgtttccttttatattgctattcaaaatcaataccaccaacaaaatatttctaagtagacacttttccagacctttgtttttttgtgtcagtgtccaagctgcagataggattttgtaatacttctggcagcttctttccttgtgtacataatatatatatatacatatatatatatatttttaatcagaagttatgaagaacaaaaagaaaaaataaacacagaagcaagtgcaataccacctctcttctccctctctcctagggtttcctttgtagcctatgtttggtgtctcttttgacctttaccccttcacctcctcctctcttcttctgattcccctcccccccttttttaaagagtttttctcctttctcaaggggagttaaactagcttttgagacttattgcaaagcattttgtatatgtaatatattgtaagtaaatatttgtgtaacggagatatactactgtaagttttgtactgtactggctgaaagtctgttataaataaacatgagtaatttaacaccaaaaaaaaaaaa

SEQ ID NO: 28 - DNA, 1339 bp, Homo sapiens
  misc_feature: Incyte ID No 1329044CB1
cacacattttgaaatatgtgtcaaatatttaggaatactaatttaatctaaaaatccataattgaaatttaaagagttaaaagtacacaaaatagactgaaaaattaattatccaacaatatgaaaaccccaaatgacctatttctcagacaactagggtatctttcaatctgctgctttgtattttcttctgaagagtcaaaaaattataaaatatccttaatagtctacttgacatttttgactatggaaaccaagcccaggaatagtatatacagtgtactaacccagtcaacccacccagactttgagtccccacgtacaggagtccccaaccccagggccgaagaccagtaccagtttgaggcctattaccgggtcacatagcaggaggtgagcagcgggatgaggcagcattactgcctgaactctgcctcctgttagatcagcagcagcattaggttctcataggagtgtgaaccctattgtgaactgtgcaggtgagggatctaggttgcatgctccttatgagaatctaatgcctgatgatccgagtgggacagtttcatcccaaaaccatccaccaccaccaccctctcccatgtccatggaaaaactgtctttgacaaaactggtccctggtgccaaaaaggttggggaccgctgcctaagtagaccaagtctatgagtatctaaacccagcagacaaagtacacattcacatccaaaaaatgaagagcaggagtaagatgagcagagcatcagtgaggctttgggaaggtcaactattagcatccattttgatatgctggcatttcattggatattggacattcacagcatcattctcttccaggaagggagaagagctgtggagtagatctgtgagcaaaagagaaatggaagacagtggctgatcccaaatacatttgagtagcagataattaagaagagttatacaggccagagacaacgaacacaaagaatctaacagtctacccaaaaattatgccctaaaacagtgacttctcaaccagactcaatttctctgcaatgtctggagacattcagtagttgtctggagacattcagtagttgtcacaactgagctggaggtactgtgttgctcctggcatttagtaggtagagatcagggatgctgctaaacaccctacagtgtacaggacagcccctacaacaaagaattaaccaaaatgtcaacaatgctaaggctgagaaactctgacctaaaatgacaatcattatgactaaccatgtgcatagctgaaaagatccatgaaaagccttaaaatagatcgcaataaacattatgtagtca

SEQ ID NO: 29 - DNA, 987 bp, Homo sapiens
  misc_feature: Incyte ID No 1493630CB1
aaatgtgcatagcagaatgttaacagacgctgcctttagggagagataaaaagcataatgacattagctaggaaagttaattttcagttcttactgaagtgctgtatgaaactgaaatttccaaggaactgaattttgtgagccaaatgagcatgcaattcttgtttaagatggtggccttatgctgttgtctctggaagatctccggctgtgaggaagtccctctaacttacaacctgctcaagtgcctcctagataaagcgcactgtgtactcctgacaccttgtggttacatcttttccttgatcagtccagaaattctcaaactcactttaatcactttgcagatcctcttaatactcaaaaatctacacttactgtggctgacagtttcaagcagatgtgttcatcgcagtagtgcaagaaaagaaaagtagaagaaccctgcagagatttgatggaacccagcttctattcattaaaaccaatggcaaaatataaagcaaataggaggtgacgaaggttacaaagatacgtattgtttatgttttccctggggtgtgctgattgtcaggcatcagttccctgtgccattcattccccaacacagcatgcatcagaaattttatcaataaatgctttctctctcaatgttcaacctatgctgatagaccattaaatacagtttttgggttcacagcttgtcatcatcatttgtctatacatgtggaaaagaatatctaataagatactctcagcattttgcacacttaaactaagatgctgaatgctgtattttacggaataatcagccacattaaatttggagactcaacaagcatgctgtgaacattcaacattaggtttaaattttatttttaaaagttaataataaaaggatatatgttaagtattatgaaaccctgcatatactgtaataaaatggtggatgtgaatggacaatatatgcaataaaatatataaaaa

SEQ ID NO: 30 - DNA, 842 bp, Homo sapiens
  misc_feature: Incyte ID No 1533041CB1
ggtgcctcctgcagtttgggagacatggaccagcatctggtcttgtttccaggagcatagaagccacatcgttgagacatcaggaaggtaaaaacccagcggcttagccaagccctaagcctgtccccagaccaaccctgggacctatacagaacagagggccagagctagggctgctgcttctgctccagcccctttgcctctgtcctcccatcccctcaacaccctgcttctcccggggacgcttttgagtgggccctgcccggggagctgcagagcagcagcacctttctctgagaagaggtccttggttgggtcaaggacagggctgagcgtggaagggggaggagtcaggggctctgtgttaggatgcggctttctctgcctctgggcagcctgctttggcctttccttgtatgtgggtgtttattacaagtggctttgtgtcagacacgctcggctccccacctggacacacactcaccagtggcctttcagtgtagcgggaggaagccggtgtccctggatgtgaagctcacactgatgggctggggcaggggcctgggccggcgagggggccggggggaggggacagagctgaggatttcctggagtgccctgcaggcacagaggaggtcagcaaaggtcttgaatagattttctctggaaataaagaatccttagatgcctaaaaattcccttcctgttccctcctggtcctgggacacctcccaggggactgttccttatttctctctcctggtgtgggtaaagggacagttacaaaccaggtcaccatcctcagaggctgagccctgtacccacccagcacagccactccg

SEQ ID NO: 31 - DNA, 1125 bp, Homo sapiens
  misc_feature: Incyte ID No 1566162CB1
gtaagaatcccagatccatagtttgggtcgggtaaaggtttgatgagatgataagggtggtttatttcagactttgggtaaaagggagtatgaatcagttcattaattcatcttgtttcccagagagaaaaaaaagatcaagagaagccattctggctctgccacatccccacagccagccctgttttatttcaactgcttgctcaaattaaactcaccacactggagtgcaatctgccaggagccagctggggtgtattgtgttcagttgttcaaatgttcaggcccgttctgggtaagttcatgctggtgtctcttggagggatgcctagggtgaagggctgagctctgaagtcagaatgatctgttttagaacctggctctttcatagattataggtcatttcctctttctttatggcaactcacaaaagaggaatcaggaaagctcctgtaccaatgtggctctctaacaggatctggaactgaagactaattatttggatgtggcttacactcaaaaggacattttgaagtggttgaagaggagaaactttcctaacaacttgttcaaagactcttttactccagggaacatagctaactgggaagagggtggaggatctagtgccttgtcccatactggaaacacacaggacagaaggctccacaacacagcctggccttgggaggaaggtaggaggttctgactcagcagccagctgtgagaggtggaagaggacccttgatctgggcaagcaagggttcagtcctgctggaaagatgactcttttaccaagagaatactgaatcccagagaagtctcagactgcagtactctaggagtgaaaaccagttggatgtctagaggaactcagccagctggataagttctcatctctccacctttgacattgtgcttcacactgatgttgatgttcatcaagggtttgtcttccaccctcttcttgggcagcacactctcccacagggatcccatctgtttttatagcttccatttccatctctatctgttgcctcatgctgtttccccagtgacaaactcaatatacaattacctgctgggactctacctggatacctgcacatgagacccaacaaagga

SEQ ID NO: 32 - DNA, 597 bp, Homo sapiens
  misc_feature: Incyte ID No 1811831CB1
cttatttgaaaatcaatataaccacatctataaaaacaattttaaagaatgcccaaatcccagagccatcatctcacccaattacaactcttgccttcatgtctgcttggcctcttgccccctgttcctgctgtgcatgcatacattttacagggatgtgtcctcagtggacgtgagatttttttttctgttttgcagttttttacacaaacattttcatttgttgttcctgtttttccttctttcccaggaggctttaggctcccattctcctctccctggctttctctctgtcccatccaccgctctacccttcaggcctgcctatatgaaagaggtctctttctatgcagaaaactcactttaacaagatgtgggtgctcttacacagacctcatatgaggaaaatagcacatcagtgaacttggggtccctgggagtcacagtgatgttcaccagaaaatcagacaacggtaatgtacctcccccatcaggttgccaaaaattagaataggttttgtgttttttggtgttgtttgtttgagacggagtcttgctctgtcaccaggctggagtgcagtggtgcgatctcggctc

SEQ ID NO: 33 - DNA, 658 bp, Homo sapiens
  misc_feature: Incyte ID No 1835447CB1
acgatgcgagccaaaggattcttggctccaagcctggtcctggctgttagtttggaactcatgcacccagatgctaactcgccctcagaatgcagaggggatgaaacactgaccggacaattcaatctgtatatgggagataaactggaggggaagacgaatggcaggagggtgaagaggaaactgaattactgtgcaaacacccgccactcaaatccgggtggttactgcagagtgaataacgataggtactatttcgtgtaaggcaaagtcctttgaaagggctcctagagcgtcaaggcctccacctgatgaatgaatgagtcaggcaggcccagctccacttcacggatgggaaaactgaggtacgaggcctcgctgaaagatgcgaggcagagcggagaaccagaagcaccacttctctcaggctgatgctctaatctcggctccccccgcccctacaatggcgtagacggcctccgccgcccgactcacacacaccctcccccgggaacggcaagtctcctcgggttccaaggacagggtcaaaagacaagaggcccgaggcgctcccgccgtgatttgcagccagataccgttgggagcgcacgcagagagcgttgggagcgtgcgtacctccagcccaacatggcggcgg

SEQ ID NO: 34 - DNA, 639 bp, Homo sapiens
  misc_feature: Incyte ID No 3892281CB1
gggttacaggcgtgagcacggtgcacggcctgctttataacaaattgtccccaaacttagcaacaactatttgccccagacaactgtttttcccccttccttctatggattgaccagccagttctgctttagatggtattggatggagccctggaatggctgaaaagtccaaactggcctgacttgctcagccagcaatgcggtgcaggctcctagctggggccttagttctcctgcacctgcggctctccatatggctgcttggacttcctcatagcatggcagatggattacgagaaggagcattcccaaataaaggaccacataagctagatctctggagggctagcctcagaagtcacccagtgtcacatgggccacattttattggttacagggccagccagtttgaaggggaagagaaatatgtcgctgtttatgctgtgtccagtgctagcttgctacctgctctcccagttccagtgctcagggcagcactggcagaacagatgtacttactgagttaaaaacagcaacatccaagacaattgttaacttttaaaactgtctcccatcccagaaggtataactaaaaaactaacaataaaaataatagtaataaataataaaaaaaaaaaa

SEQ ID NO: 35 - DNA, 996 bp, Homo sapiens
  misc_feature: Incyte ID No 4318494CB1
gtctgactatctgatggagacaccttctgagccgaaacagtgtggccatagtggctgtgtcctcagagatgaggagcccctccttccctttcacattgctctctggccttcccggacctggcttctcgcagctttgtgtgcgtgtttctcaggtgtccaggaatcccatgcgaagtgaaggctgctttggtctcctcaagtctgtccaggacaatccagcctctgccctggaactgctggatttctcagatatccaggtgaacgcagagtttgatggccttgctagctcagtgaggggaattcttccagaactctgcataaagactggcgcttgcagagtggagtataaaaaggagttgctgccagtcttcagatcagccctgccagcgtctgtccctaagtgaccttggagtgtggcttcctcatctccaatcagctgctttgacctccagggtatatacttgaaagaaatgaagacatatgtccccacaagaactcgtgcacgaatatccatagcaacattatttataatattctaagagtgaaaatgcccaccagtggataaatgcaatgtggtatatccatacagtggaatattatttggcaataaaaaggaatttgaggtgataccaatgttctaaaatgtattgtggtgatggctacgtaactgtgcatattctaaaggcaattgaattacagatgctttacatgaatgaaccgtatggtatgtgaacggcatctcaataaaactgtttcgaaaagaaggaaaaggacggacacatgctgaaaacgggtgaaactagaaaacatggcgctaagtgaaagaagccagccacaagatcacgtgtcgcatgaccgcatttatgtgaaacatccggagtatgcaaatctatacagacagaaagtagattatacattgcctaggtgcagagaaatggaagtattggaggttgacggctaaaggatgtggatttctttgggggtgataaaagtggt

SEQ ID NO: 36 - DNA, 795 bp, Homo sapiens
  misc_feature: Incyte ID No 5090841CB1
ggatggggtcagcacccagaagccagccccctctgacagcttcctctttggccaagccctgcctctgtacagcctcgagtggacagccagaggctgcagctggagcccagagcccaagatggagccccagctggggcctgaggctgccgccctccgccctggctggctggccctgctgctgtgggtctcagccctgagctgttctttctccttgccagcttcttccctttcttctctggtgccccaagtcagaaccagctacaattttggaaggactttcctcggtcttgataaatgcaatgcctgcatcgggacatctatttgcaagaagttctttaaagaagaaataagatctgacaactggctggcttcccaccttggactgcctcccgattccttgctttcttatcctgcaaattactcagatgattccaaaatctggcgccctgtggagatctttagactggtcagcaaatatcaaaacgagatctcagacaggagaatctgtgcctctgcatcagccccaaagacctgcagcattgagcgtgtcctgcggaaaacagagaggttccagaaatggctgcaggccaagcgcctcacgccggacctggtgcaggactgtcaccagggccagagagaactaaagttcctgtgtatgctgagataacaccagtgaaaaagcctggcatggagcccagcactgagaacttccagaaagtgttagccttctcccaactgtgttataccaaccacattttcaaatagtaatcattaaagaggcttctgcatcaaaaa

SEQ ID NO: 37 - DNA, 1419 bp, Homo sapiens
  misc_feature: Incyte ID No 2006548CB1
tggcctccccagcttgccaggcacaaggctgagcgggaggaagcgagaggcatctaagcaggcagtgttttgccttcaccccaagtgaccatgagaggtgccacgcgagtctcaatcatgctcctcctagtaactgtgtctgactgtgctgtgatcacaggggcctgtgagcgggatgtccagtgtggggcaggcacctgctgtgccatcagcctgtggcttcgagggctgcggatgtgcaccccgctggggcgggaaggcgaggagtgccaccccggcagccacaaggtccccttcttcaggaaacgcaagcaccacacctgtccttgcttgcccaacctgctgtgctccaggttcccggacggcaggtaccgctgctccatggacttgaagaacatcaatttttaggcgcttgcctggtctcaggatacccaccatccttttcctgagcacagcctggatttttatttctgccatgaaacccagctcccatgactctcccagtccctacactgactaccctgatctctcttgtctagtacgcacatatgcacacaggcagacatacctcccatcatgacatggtccccaggctggcctgaggatgtcacagcttgaggctgtggtgtgaaaggtggccagcctggttctcttccctgctcaggctgccagagaggtggtaaatggcagaaaggacattccccctcccctccccaggtgacctgctctctttcctgggccctgcccctctccccacatgtatccctcggtctgaattagacattcctgggcacaggctcttgggtgcattgctcagagtcccaggtcctggcctgaccctcaggcccttcacgtgaggtctgtgaggaccaatttgtgggtagttcatcttccctcgattggttaactccttagtttcagaccacagactcaagattggctcttcccagagggcagcagacagtcaccccaaggcaggtgtagggagcccagggaggccaatcagccccctgaagactctggtcccagtcagcctgtggcttgtggcctgtgacctgtgaccttctgccagaattgtcatgcctctgaggccccctcttaccacactttaccagttaaccactgaagcccccaattcccacagcttttccattaaaatgcaaatggtggtggttcaatctaatctgatattgacatattagaaggcaattagggtgtttccttaaacaactcctttccaaggatcagccctgagagcaggttggtgactttgaggagggcagtcctctgtccagattggggtgggagcaagggacagggagcagggcaggggctgaaaggggcactgattcagaccagggaggcaactacacaccaacctgctggctttagaataaaagcaccaactgaaaaaaaaaa

SEQ ID NO: 38 - DNA, 1265 bp, Homo sapiens
  misc_feature: Incyte ID No 2207183CB1
gtttactgagggcagatggaggggcccgagtttctgcgaaccgcgacctcggcgtccggacgcggggaacaccgggctgagggagtctgcagtcggctccgggaagccgcgcggcgacgggggaggccttcactaaaggggaaaaggaagagggggtcggccagtatccccgaaagagggctagggcgcatgaagaccagcgcagagctccacgagcaggaaaagcccccaagcagccccagggcgactggaccgggccgcttaggccacgcccggggaagagggcctgacgcgctgcggggcggggccgcggggccgggtcgcgcgagcagcggagcaccaagggaacggaaaatggcgcctcacggcccgggtagtcttacgaccctggtgccctgggctgccgccctgctcctcgctctgggcgtggaaagggctctggcgctacccgagatatgcacccaatgtccagggagcgtgcaaaatttgtcaaaagtggccttttattgtaaaacgacacgagagctaatgctgcatgcccgttgctgcctgaatcagaagggcaccatcttggggctggatctccagaactgttctctggaggaccctggtccaaactttcatcaggcacataccactgtcatcatagacctgcaagcaaaccccctcaaaggtgacttggccaacaccttccgtggctttactcagctccagactctgatactgccacaacatgtcaactgtcctggaggaattaatgcctggaatactatcacctcttatatagacaaccaaatctgtcaagggcaaaagaacctttgcaataacactggggacccagaaatgtgtcctgagaatggatcttgtgtacctgatggtccaggtcttttgcagtgtgtttgtgctgatggtttccatggatacaagtgtatgcgccagggctcgttctcactgcttatgttcttcgggattctgggagccaccactctatccgtctccattctgctttgggcgacccagcgccgaaaagccaagacttcatgaactacataggtcttaccattgacctaagatcaatctgaactatcttagcccagtcagggagctctgcttcctagaaaggcatctttcgccagtggattcgcctcaaggttgaggccgccattggaagatgaaaaattgcactcccttggtgtagacaaataccagttcccattggtgttgttgcctataataaacacttttttcttttaaaaaaaaaaaaa

SEQ ID NO: 39 - DNA, 1720 bp, Homo sapiens
  misc_feature: Incyte ID No 2267403CB1
cccacgcgtccgcgcctctcccttctgctggaccttccttcgtctctccatctctccctcctttccccgcgttctctttccacctttctcttcttcccaccttagacctcccttcctgccctcctttcctgcccaccgctgcttcctggcccttctccgaccccgctctagcagcagacctcctggggtctgtgggttgatctgtggcccctgtgcctccgtgtccttttcgtctcccttcctcccgactccgctcccggaccagcggcctgaccctggggaaaggatggttcccgaggtgagggtcctctcctccttgctgggactcgcgctgctctggttccccctggactcccacgctcgagcccgcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcggcctcacaacctgccccgaaccaggctgcccagcacccctcccactgccagactcctgctgccaagcctgcaaagatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacataagaaagcctgtgtgcatggcgggaagacgtactcccacggggaggtgtggcacccggccttccgtgccttcggccccttgccctgcatcctatgcacctgtgaggatggccgccaggactgccagcgtgtgacctgtcccaccgagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaagatgaggaaactgaggctcagagaggtgaagtacctggcccaaggccacacagccagaatcttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgttgttattatatattaataaataagaagttgcattaccctcaaaaaaaaaa

SEQ ID NO: 40 - DNA, 1055 bp, Homo sapiens
  misc_feature: Incyte ID No 2933038CB1
gagaaaaacaacaggaagcagcttacaaactcggtgaacaactgagggaaccaaaccagagacgcgctgaacagagagaatcaggctcaaagcaagtggaagtgggcagagattccaccaggactggtgcaaggcgcagagccagccagatttgagaagaaggcaaaaagatgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccctaaaggcagcagctcaaggatggcactcagatctccatggcccagcaaggccaagataaatctaccaccccaggcacctgtgagccaacaggttaattagtccattaattttagtgggacctgcatatgttgaaaattaccaatactgactgacatgtgatgctgacctatgataaggttgagtatttattagatgggaagggaaatttggggattatttatcctcctggggacagtttggggaggattatttattgtatttatattgaattatgtacttttttcaataaagtcttatttttgtggctaaaaaaaaaa

SEQ ID NO: 41 - DNA, 1379 bp, Homo sapiens
  misc_feature: Incyte ID No 3216587CB1
cgggtcctcgcgcggggaagcggttccgaaggctcgcggggagcggctagccctgagtccctgcatgtgcggggctgaagaaggaagccagaagcctcctagcctcgcctccacgcttgctgaataccaagctgcaggcgagctgccgggcgcttttctctcctccaattcagagtagacaaaccacggggatttctttccagggtaggggaggggccgggcccggggtcccaactcgcactcaagtcttcgctgccatgggggccgtcatgggcaccttctcatctctgcaaaccaaacaaaggcgaccctcgaaagataagattgaagatgagctggagatgaccatggtttgccatcggcccgagggactggagcagctcgaggcccagaccaacttcaccaagagggagctgcaggtcctttatcgaggcttcaaaaatgagtgccccagtggtgtggtcaacgaagacacattcaagcagatctatgctcagtttttccctcatggagatgccagcacgtatgcccattacctcttcaatgccttcgacaccactcagacaggctccgtgaagttcgaggactttgtaaccgctctgtcgattttattgagaggaactgtccacgagaaactaaggtggacatttaatttgtatgacatcaacaaggacggatacataaacaaagaggagatgatggacattgtcaaagccatctatgacatgatggggaaatacacatatcctgtgctcaaagaggacactccaaggcagcatgtggacgtcttcttccagaaaatggacaaaaataaagatggcatcgtaactttagatgaatttcttgaatcatgtcaggaggacgacaacatcatgaggtctctccagctgtttcaaaatgtcatgtaactggtgacactcagccattcagctctcagagacattgtactaaacaaccaccttaacaccctgatctgcccttgttctgattttacacaccaactcttgggacagaaacaccttttacactttggaagaattctctgctgaagactttctatggaacccagcatcatgtggctcagtctctgattgccaactcttcctctttcttcttcttgagagagacaagatgaaatttgagtttgttttggaagcatgctcatctcctcacactgctgccctatggaaggtccctctgcttaagcttaaacagtagtgcacaaaatatgctgcttacgtgcccccagcccactgcctccaagtcaggcagaccttggtgaatctggaagcaagaggacctgagccagatgcacaccatctctgatggcctcccaaaccaatgtgcctgtttctcttcctttggtgggaagaatgaga

SEQ ID NO: 42 - DNA, 702 bp, Homo sapiens
  misc_feature: Incyte ID No 5037143CB1
ggcaggtgctcgcttggtctagtgcccatttactctggactccggatggctgccgcacgcctctgcctctccctgctgctcctgtccacctgcgtggctctgttactacagccactgctgggtgcccagggagccccactggagccagtgtacccaggggacaatgccacaccagagcagatggcccagtatgcagctgatctccgtagatacatcaacatgctgaccaggcctaggtgtgtgccacagttggggagagagatcccagcccctgggaccctgggcccactccacattcctggccacaccctatccccagccccagccccagccccttccaggcctgctcttgggaaaacagggcatctgtgctcaacaggcctagaccaatgtgccctgggcaagatggtgcctacaggcagatatgaaacaggtgggctggcacctgggcacagtgcttgcccctgctgcctcttccctcccaggtatgggaaaagacacaaagaggacacgctggccttctcggagtgggggtccccgcatgctgctgtccccagggagctcagcccgctggacttataatgccaccttctgtctcctacgactccatgagcagcgccagcccagctctcccctctgcacccttggctctggccaaagcttgcttcctgctcccacacagatcaataaagaagcatgtcc

SEQ ID NO: 43 - DNA, 1855 bp, Homo sapiens
  misc_feature: Incyte ID No 1235265CB1
acctgggtccggccccctgaggccgcccggactccaggctcagacaaggagcggcctgtggagcggagggagccctccatcaccaaggaggagaaggacagggacctccccttctcacggccccagctccgagtttctcctgctactcccaaggcccgggctggtgaggaggggcctcggccaaccaaggaatctgtgcgggtaaaggaagagcggaaggaggaggctgccgccgccgctgccgctgctgctgccgccgccgctgccgccgccgcagcagccactgggccccagggccttcacctgctgtttgagaggccccggccgcccccgtttctgggccctagcccaccagatcgctgtgctggcttcctggagccaacctggttggcagcacccccacgcctggcaaggccaccccgcttctatgaggcgggtgaggagctaactggacccggggccgtggccgctgcccgcctctacggtctggaacctgctcaccccttgctctacagccgcttggctcctccaccaccacctgctgcggccccgggaacccctcaccttctcagcaagaccccaccgggagcccttttgggggcaccacctccgcttgtgcccgccccccggcccagttccccacctaggggccctggcccagctcgggctgacaggtgaggggaacgggggggggtcggggcaaagctccatctccccttcctttaaccaggtcctagggctgaggttttaagccagggctggagggcaaaggtcataacctcaccagccacctctgaggtcatggaacctgggaacagaagcctcaacccccacaagaccaagcatcacatggagtgtagggtcactgggagagcagaggtcacagcctctagagaagggagaggggcgtgtgcatgggagtgtggctcatctcgggggccatggggcctcctgaggtacacctttgcccctgtaagggcctctaggccctgggcctgcctccccaagggctcactaagccagaggccaaagttgccccctcccgttcacctaccacccaagtcctcatgccctccgagggctgggggaggaggggctcaaggaaggggggttccatgtacatatttatcacccctttcacatagccccaagaccttttgtacatttttacaggggtgcccctcccaacagttcccttcctggttaattaaaccctcagactggtgctgtgttcctagcctctggcctctctgtggggaaaggggactgcagggggaagagccgggaagggacagtcaggcttctccctgggaaggtggggccagcaggagatgaccaacagggggcaggacctggggacctgggctggagggaagggcagaagcttcctacttggctgacagccccggttcccccaacatgttcccgttcactctgcccccacccccaaaggctcagcctctaaatctcagactccaccacctcttaatggctcagtccccttcaccccatttccaagtgcccccaggactcctgggccctgcttccctgaaccctgttctccaaaaccctgccccaggctaagggtggccagagaaggtcaccatgtaccacacaccaaagaagggggtcggcccaggggtgggcgacacaggcagcttcttcggcagcctcacggcagcaaccccagccttcccaaagcagcaggcgcctccaggctggggcccaacctagaaggcaggggtcaatctaacaaaaccctaacgttgacttttttccctggtggggcttcttctgtaacatgacttgcgaatatttatataaaaacgagtgttacaatgagaaaaaaaaaaaa

SEQ ID NO: 44 - DNA, 1132 bp, Homo sapiens
  misc_feature: Incyte ID No 5571181CB1
acaggttctccttccccagtcaccagttgctcgagttagaattgtctgcaatggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatttgaccagagcaaagctgaaaaatgaataactaaccccctttccctgctagaaataacaattagatgccccaaagcgattttttttaaccaaaaggaagatgggaagccaaactccatcatgatgggtggattccaaatgaacccctgcgttagttacaaaggaaaccaatgccacttttgtttataagaccagaaggtagactttctaagcatagatatttattgataacatttcattgtaactggtgttctatacacagaaaacaatttattttttaaataattgtctttttccataaaaaagattactttccattcctttaggggaaaaaacccctaaatagcttcatgtttccataatcagtactttatatttataaatgtatttattattattataagactgcattttatttatatcattttattaatatggatttatttatagaaacatcattcgatattgctacttgagtgtaaggctaatattgatatttatgacaataattatagagctataacatgtttatttgcctcaatgccct

SEQ ID NO: 45 - DNA, 1906 bp, Homo sapiens
  misc_feature: Incyte ID No 685374CB1
cgaggcaagaattcggcacgaggggaccagcttataaagaagcatggctttgttaaggaagtcgtattcagagcctcagcttaagggtatagttaccaagctatacagccgacaaggctaccacttgcagctgcaggcggatggaaccattgatggcaccaaagatgaggacagcacttacactctgtttaacctcatccctgtgggtctgcgagtggtggctatccaaggagttcaaaccaagctgtacttggcaatgaacagtgagggatacttgtacacctcggaacttttcacacctgagtgcaaattcaaagaatcagtgtttgaaaattattatgtgacatattcatcaatgatataccgtcagcagcagtcaggccgagggtggtatctgggtctgaacaaagaaggagagatcatgaaaggcaaccatgtgaagaagaacaagcctgcagctcattttctgcctaaaccactgaaagtggccatgtacaaggagccatcactgcacgatctcacggagttctcccgatctggaagcgggaccccaaccaagagcagaagtgtctctggcgtgctgaacggaggcaaatccatgagccacaatgaatcaacgtagccagtgagggcaaaagaagggctctgtaacagaaccttacctccaggtgctgttgaattcttctagcagtccttcacccaaaagttcaaatttgtcagtgacatttaccaaacaaacaggcagagttcactattctatctgccattagaccttcttatcatccatactaaagccccattatttagattgagcttgtgcataagaatgccaagcattttagtgaactaaatctgagagaaggactgccaaattttctcatgatctcacctatactttggggatgataatccaaaagtatttcacagcactaatgctgatcaaaatttgctctcccaccaagaaaatgtaaaagaccacaattgttcttcaaaaacaaacaaaacaaaacaaaacaaaattaactgcttaaatgttttgtcggggcaaacaaaattatgtgaattgtgttgttttcttggcttgatgttttctatctacgcttgattcacatgtactcttttctttggcatagtgcaactttatgatttctgaaattcaatggttctattgactttttgcgtcacttaatccaaatcaaccaaattcagggttgaatctgaattggcttctcaggctcaaggtaacagtgttcttgtggtttgaccaattgtttttctttctttttttttttttttagatttgtggtattctggtcaagttattgtgctgtactttgtgcgtagaaattgagttgtattgtcaaccccagtcagtaaagagaacttcaaaaaattatcctcaagtgtagatttctcttaattccatttgtgtatcatgttaaactattgttgtggcttcttgtgtaaagacaggaactgtggaactgtgatgttgtcttttgtgttgttaaaataagaaatgtcttatctgtatatgtatgagtcttcctgtcattgtatttggcacatgaatattgtgtacaaggaattgttaagactggttttccctcaacaacatatattatacttgctactggaaaagtgtttaagacttagctaggtttccatttagatcttcatatctgttgcatggaagaaagttgggttcttggcatagagttgcatgatatgtaagattttgtgcattcataattgttaaaaatctgtgttccaaaagtggacatagcatgtacaggcagttttctgtcctgtgcacaaaaagtttaaaaaagttgtttaatatttgttgttgtatacccaaatacgcaccgaataaactctttatattcattcaaagaaaaaaaaa

SEQ ID NO: 46 - DNA, 1803 bp, Homo sapiens
  misc_feature: Incyte ID No 843193CB1
caactggccaggagcctctgttacattgtggctaaggagctgcctgccaggggcagccattggggccaccgctgatagtgcctgtcctcttggtactgcctctgcctccctccgctaaggaggcaccttgcctgcctgctgtcccatagtgcccagccccagccccagccccagctccagcccatagaggagggaggaacactggaagggccctgagcaccagggggcaaggccgggaagaagatgggtatgagctcaggattccacagttagtgcttcaaagaaatgctcacgggaccctgcaggagctttcagagtcccccacatgctctctggtgaccctaactcgcagcaccatctgctctgtgcccatgtgctgggcaaggggtctttcaaggccagtggggaggatgaggaaggaatctggttgtcccggctaatggagcatgtccttggagttctgggggagatgacaggctctggtctaagaggtagggacaggggttctgtccctaatgagctgtgtgccccgtgcacctccttcatagaatacgaggacgggatagaaccctgagggctccttccagctcccagagtcctgattccagggctgtgctctgtcaataagtgtcccccagcctgggcagaccccagtcccttctgtaaggtagacgcaaagcaaagaggttatgaccggctcacccaggggcctgggaaggctatggccatatgcccacttcactctgcaggacaagtggcctgtccccactatattcacctcctcacccctctcccttggatggaccagtggtggtgtcacccaaagcaaattgacactatttttcccttggtaaccgcaaagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttgaccaaccacgccgagaagcagaagcagttcgagtttgtgggctccgcccccacccgccgggcgaagcgcacacggcggccccagcccctcacgtagtctgggaggcagggggcagcagcccctgggccgcctccccacccctttcccttcttaatccaaggactgggctggggtggcgggaggggagccagatccccgagggaggaccctgagggccgcgaagatccgagcccccagctgggaaggggcaggccggtgccccaggggcggctggcacagtgcccccttcccggacgggtggcaggccctggagaggaactgagtgtcaccctgatctcaggccaccagcctctgccggcctcccagccgggctcctgaagcccgctgaaaagtcagcgacttaaggccttgcagacaaccgtctggaggtggctgtcctcaaaatctgcttctcggat

SEQ ID NO: 47 - DNA, 3053 bp, Homo sapiens
  misc_feature: Incyte ID No 1359783CB1
ctagtattctactagaactggaagattgctctccgagttttgttttgttattttgtttaaaaaataaaaagcttgaggccaaggcaattcatattggctcacaggtatttttgctgtgctgtgcaaggaactctgctagctcaagattcacaatgttgaaagcccttttcctaactatgctgactctggcgctggtcaagtcacaggacaccgaagaaaccatcacgtacacgcaatgcactgacggatatgagtgggatcctgtgagacagcaatgcaaagatattgatgaatgtgacattgtcccagacgcttgtaaaggtggaatgaagtgtgtcaaccactatggaggatacctctgccttccgaaaacagcccagattattgtcaataatgaacagcctcagcaggaaacacaaccagcagaaggaacctcaggggcaaccaccggggttgtagctgccagcagcatggcaaccagtggagtgttgcccgggggtggttttgtggccagtgctgctgcagtcgcaggccctgaaatgcagactggccgaaataactttgtcatccggcggaacccagctgaccctcagcgcattccctccaacccttcccaccgtatccagtgtgcagcaggctacgagcaaagtgaacacaacgtgtgccaagacatagacgagtgcactgcagggacgcacaactgtagagcagaccaagtgtgcatcaatttacggggatcctttgcatgtcagtgccctcctggatatcagaagcgaggggagcagtgcgtagacatagatgaatgtaccatccctccatattgccaccaaagatgcgtgaatacaccaggctcattttattgccagtgcagtcctgggtttcaattggcagcaaacaactatacctgcgtagatataaatgaatgtgatgccagcaatcaatgtgctcagcagtgctacaacattcttggttcattcatctgtcagtgcaatcaaggatatgagctaagcagtgacaggctcaactgtgaagacattgatgaatgcagaacctcaagctacctgtgtcaatatcaatgtgtcaatgaacctgggaaattctcatgtatgtgcccccagggataccaagtggtgagaagtagaacatgtcaagatataaatgagtgtgagaccacaaatgaatgccgggaggatgaaatgtgttggaattatcatggcggcttccgttgttatccacgaaatccttgtcaagatccctacattctaacaccagagaaccgatgtgtttgcccagtctcaaatgccatgtgccgagaactgccccagtcaatagtctacaaatacatgagcatccgatctgataggtctgtgccatcagacatcttccagatacaggccacaactatttatgccaacaccatcaatacttttcggattaaatctggaaatgaaaatggagagttctacctacgacaaacaagtcctgtaagtgcaatgcttgtgctcgtgaagtcattatcaggaccaagagaacatatcgtggacctggagatgctgacagtcagcagtatagggaccttccgcacaagctctgtgttaagattgacaataatagtggggccattttcattttagtcttttctaagagtcaaccacaggcatttaagtcagccaaagaatattgttaccttaaagcactattttatttatagatatatctagtgcatctacatctctatactgatcagatcttgtgagacttattcactaccacaacaatagtatgggggaaactgcccccatgattcaaattatctccctcccacaacacgcgggaattatgggagtacaattcaagaggcgatttgggtggggacacagccaaaccatatcagtgtatatgtagcacattttctttgtggatgtggatccttgagggtggaggaaggagcaagggactgattattttgagttggtggtagagcctggtcgtcaggaaagaccacataggggtgcagcataaactatgacttcagagatggaaaggaattccccagatatgcaggtgaggaaggggacttgtggcaaggtccctgcatgtgtgaaggcatgggacaggagagactatcacttgtattgctcttgggtcttccctccccttccccccgcccttgcctctgtcccagggatccactggggctaaagggatgtcctggggcccagactgctagaggagccatgctacagggtctgttcagcacccccacctgactcctgacgcagagaggtggagaagctcatgggtcagcactgggcttggctgcccatctgaggcctgcaactgtggccagcatggaatgtctatgggaggccatcagctgggacatttagaactcttctgggaggggcgctcttgacccttctggagagcatgtggggaagcagaggagctgctcccctaagccaggaggagctggtgctgagttgtttattgggtgagagttgtgtccaacaccaatgatctttaaatgaactgagtcctagagctgtccggaagactagaactaggaccccggttggagactgcagggagtgcttatagttgacatcggacagggcagctccgttaggaaggagtgtcacctgcactgggaaggttccaaggaagaggttgcctgccttagagaccaagtaccctgataggccagcatcaggctggcctagtacaaagatggtctcgaagcgcccccagggaaatgtgcctccaacaaatcgaagtggataaaaagggcaggacactctaatgagcaccgggcactctctagacatctttttcagattccccctcgctatgaggcaggtctgtctccatcttgcagatgagaatctcagtgaggaggttcaggatcacacagccagtacaggactctggtgccgcgccgtctctaaagcccaccgttcaaccactcgcctgtgctctcagagaggtcggtggaacctgcgggatttctggaaggggagcctgagagagcttcagaagggcgaagactgtca

SEQ ID NO: 48 - DNA, 560 bp, Homo sapiens
  misc_feature: Incyte ID No 1440015CB1
cccacgcgtccgcccacgcgtccgaaaaggatcgaaggcagccccggagcccagcggccgggaggcgcgcccgaacgaagccgcggcccgggcacagccatggcccggcgggcggggggcgctcggatgttcggcagcctcctgctcttcgccctgctcgctgccggcgtcgccccgctcagctgggatctcccggagccccgcagccgagccagcaagatccgagtgcactcgcgaggcaacctctgggccaccggtcacttcatgggcaagaagagtctggagccttccagcccatccccattggggacagctccccacacctccctgagggaccagcgactgcagctgagtcatgatctgctcggaatcctcctgctaaagaaggctctgggcgtgagctcagccgccccgcaccccaaatccagtacaggaggctgctggtacaaatacttgcagaaatgacaccaataatggggcagacacaacagcgtggcttagattgtggccaaccccagggaaaggtgctgaattgggaaccttgttgaatgggccccatt

SEQ ID NO: 49 - DNA, 613 bp, Homo sapiens
  misc_feature: Incyte ID No 1652885CB1
ctcgagcgcgggggctgtgctgaagggccaggaggccagcaggaagaccagctctccgcggtgagtgtgtgtcccatccccatatcaccattgcctctacttcggttgagacttgtgctctaggttctgatactttctctggctgccaaggttgtcattaggtcctcacatctgaggaaatggttccgcagcctcctaccacttgcccctggaagccagtcccttccccttgtgacttacgtgtccagggtatttgcccatcttccttccctgatacccccttggcacaggaggaagacagcgaacccctcccaccacaggatgcccagacctctgggtcactgttgcactacctgctccaggcaatggagagacctggccggagccaagccttcctgtttcagccccagaggtttggcagaaatacccagggatcctggaggaatgaatggctgagtccccgggctggagaggggctgaattcccagttctggagcctggctgcccctcaacgctttgggaagaagtgacatgtcatcccttgatatgtctgcatgcaaggtccacacccaaaagtgtcaatgtttgccccccaaataaaattgtctggctt

SEQ ID NO: 50 - DNA, 655 bp, Homo sapiens
  misc_feature: Incyte ID No 4003984CB1
cctggacccaagctccagccaaaaagcctctctcctccactcaggctgggaggttgctttctaggagctcaggatgcaaaggtggacactgtgggctgcagccttcctgaccctccactctgcacaggcctttccacaaacagacatcagtatcagtccagccctgccagagctgcccctgccttccctgtgccccctgttctggatggagttcaaaggccactgctatcgattcttccctctcaataagacctgggctgaggccgacctctactgttctgagttctctgtgggcaggaagtccgccaagctggcctccatccacagctgggaggagaatgtctttgtatatgacctcgtgaacagctgtgttcccggcatcccagctgacgtctggacaggccttcatgatcacagacaggtgagaaagcagtggccattgggcccccttggaagctccagccaggattctattttgatttaataagcttttcacatcagtgccaggtcacggctatgcacacagcatatagagagaaatcagacaccaagatgtcacagttacagcatgaccaatttgtgaaagacatttaatgatgtcctactaaatgatgggaacagatagcatggtcagagaaaacctgtttggctggga

SEQ ID NO: 51 - DNA, 630 bp, Homo sapiens
  misc_feature: Incyte ID No 4365383CB1
ccaggcccaagcttccccaccatgaattttgttcacacaagtcgaaaggtgaagagcttaaacccgaagaaattcagcattcatgaccaggatcacaaagtactggtcctggactctgggaatctcatagcagttccagataaaaactacatacgcccagagatcttctttgcattagcctcatccttgagctcagcctctgcggagaaaggaagtccgattctcctgggggtctctaaaggggagttttgtctctactgtgacaaggataaaggacaaagtcatccatcccttcagctgaagaaggagaaactgatgaagctggctgcccaaaaggaatcagcacgccggcccttcatcttttatagggctcaggtgggctcctggaacatgctggagtcggcggctcaccccggatggttcatctgcacctcctgcaattgtaatgagcctgttggggtgacagataaatttgagaacaggaaacacattgaattttcatttcaaccagtttgcaaagctgaaatgagccccagtgaggtcagcgattaggaaactgccccattgaacgccttcctcgctaatttgaactaattgtataaaaaccccaaacctgctcactaaaaaaaa

SEQ ID NO: 52 - DNA, 501 bp, Homo sapiens
  misc_feature: Incyte ID No 5497814CB1
gcccttcctgtccccaccatgtctgtcttgcctctgtgcgtcctgccacttctgctggcctcctgctcacacctgtccaccttcctctggcctcccagccttgcatgttgcttggaaacattggttggaattccatttagccggcaccgtagccttggcctcatccctgccccacggtgcctgccccttcccgctgcaatccccacttctctctgctctccaccattccacagcctgcattccctaccccgatgccctctgctgaaagtcctgggccatccacaggtggcatggtcaaggcagcagccactgcactttacctctgccaatgaccgtcatctctccaaggcctgccctggctgcagctggtattccagtgacagcctggttgcatttcagagacccttcccttcagggctgtgagaaggcggcagcgttcccatgtgggaaaaaggaggaggagggctgtgtccttcttactgtctctgagcagccccgccc

SEQ ID NO: 53 - protein, 179 aa, Cervus elaphus
  misc_feature: Genbank ID No gi511295
MPSSSALLCCLVFLAGVAASRDASAPSDSSCTHFSNSLPLMLRELRTAFSRVKNFFQMKDQLDSMLLTQSLLDDFKGYLGCQALSEMIQFYLEEVMPQAENHGPEIKEHVNSLGEKLKTLRLRLRRCHRFLPCENKSKAVEQVKSVFSKLQERGVYKAMSEFDIFINYIETYTTMKMKN

SEQ ID NO: 54 - protein, 193 aa, Macaca fascicularis
  misc_feature: Genbank ID No gi1841298
MHSSALLCCLVLLTGVRASPGQGTQSENSCTRFPGNLPHMLRDLRDAFSRVKTFFQMKDQLDNILLKESLLEDFKGYLGCQALSEMIQFYLEEVMPQAENHDPDIKEHVNSLGENLKTLRLRLRRCHRFLPCENKSKAVEQVKNAFSKLQEKGVYKAMSEFDIFINYIEAYMTMKIRNXXXXXXXXXXXXXXX

SEQ ID NO: 55 - protein, 178 aa, Homo sapiens
  misc_feature: Genbank ID No gi106805
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

(19) United States
(12) Patent Application Publication
(10) Pub. No.: US 2002/0187523 A1
(43) Pub. Date: Dec. 12, 2002

(54) Extracellular signaling molecules

(76) Inventors: Y. Tom Tang, San Jose, CA (US); Henry Yue, Sunnyvale, CA (US); Preeti Lal, Santa Clara, CA (US); Neil Burford, Durham, CT (US); Olga Bandman, Mountain View, CA (US); Mariah R. Baughn, San Leandro, CA (US); Yalda Azimzai, Castro Valley, CA (US); Dyung Aina M. Lu, San Jose, CA (US); Chandra Arvizu, Menlo Park, CA (US)

Correspondence Address: LEGAL DEPARTMENT, INCYTE GENOMICS, INC., 3160 PORTER DRIVE, PALO ALTO, CA, 94304, US

(21) Appl. No.: 09/965,528

(22) Filed: Sep. 26, 2001

Related U.S. Application Data
(60) Provisional application No. 60/134,949, filed on May 19, 1999.
(60) Provisional application No. 60/144,270, filed on Jul. 15, 1999.
(60) Provisional application No. 60/146,700, filed on Jul. 30, 1999.
(60) Provisional application No. 60/157,508, filed on Oct. 4, 1999.

(30) Foreign Application Priority Data
May 19, 2000 (US) PCT/US00/13975

(51) Int. Cl. C07K 14/435; A01K 67/00; C07H 21/04; C12P 21/02; C12N 5/06; C12N 1/21

(52) U.S. Cl. 435/69.1; 435/325; 435/320.1; 530/350; 435/252.3; 536/23.5; 800/8

(57) ABSTRACT

The invention provides human extracellular signaling molecules (EXCS) and polynucleotides which identify and encode EXCS. The invention also provides expression vectors, host cells, antibodies, agonists, and antagonists. The invention also provides methods for diagnosing, treating, or preventing disorders associated with expression of EXCS.

This application claims the benefit of Patent Cooperation Treaty International application Ser. No. PCT/US 00/13975, filed May 19, 2000, entitled EXTRACELLULAR SIGNALING MOLECULES, which claims the benefit of U.S. Provisional applications U.S. Ser. No. 60/134,949, filed May 19, 1999, U.S. Ser. No. 60/144,270, filed Jul. 15, 1999, U.S. Ser. No. 60/146,700, filed Jul. 30, 1999, and U.S. Ser. No. 60/157,508, filed Oct. 4, 1999. All of these applications are hereby expressly incorporated by reference herein.

### TECHNICAL FIELD

This invention relates to nucleic acid and amino acid sequences of extracellular signaling molecules and to the use of these sequences in the diagnosis, treatment, and prevention of infections and gastrointestinal, neurological, reproductive, autoimmune/inflammatory, and cell proliferative disorders including cancer.

### BACKGROUND OF THE INVENTION

Protein transport and secretion are essential for cellular function. Protein transport is mediated by a signal peptide located at the amino terminus of the protein to be transported or secreted. The signal peptide is comprised of about ten to twenty hydrophobic amino acids which target the nascent protein from the ribosome to a particular membrane bound compartment such as the endoplasmic reticulum (ER). Proteins targeted to the ER may either proceed through the secretory pathway or remain in any of the secretory organelles such as the ER, Golgi apparatus, or lysosomes. Proteins that transit through the secretory pathway are either secreted into the extracellular space or retained in the plasma membrane. Secreted proteins are often synthesized as inactive precursors that are activated by post-translational processing events during transit through the secretory pathway. Such events include glycosylation, proteolysis, and removal of the signal peptide by a signal peptidase. Other events that may occur during protein transport include chaperone-dependent unfolding and folding of the nascent protein and interaction of the protein with a receptor or pore complex. Examples of secreted proteins with amino terminal signal peptides include receptors, extracellular matrix molecules, cytokines, hormones, growth and differentiation factors, neuropeptides, vasomediators, ion channels, transporters/pumps, and proteases. The discussion below focuses on the structure and function of cytokines, which play a key role in immune cell signaling. (Reviewed in Alberts, B. et al. (1994) Molecular Biology of The Cell, Garland Publishing, New York, N.Y., pp. 557-560, 582-592.)

Intercellular communication is essential for the growth and survival of multicellular organisms, and in particular, for the function of the endocrine, nervous, and immune systems. In addition, intercellular communication is critical for developmental processes such as tissue construction and organogenesis, in which cell proliferation, cell differentiation, and morphogenesis must be spatially and temporally regulated in a precise and coordinated manner. Cells communicate with one another through the secretion and uptake of diverse types of signaling molecules such as hormones, growth factors, neuropeptides, and cytokines.

Hormones

Hormones are signaling molecules that coordinately regulate basic physiological processes from embryogenesis throughout adulthood. These processes include metabolism, respiration, reproduction, excretion, fetal tissue differentiation and organogenesis, growth and development, homeostasis, and the stress response. Hormonal secretions and the nervous system are tightly integrated and interdependent. Hormones are secreted by endocrine glands, primarily the hypothalamus and pituitary, the thyroid and parathyroid, the pancreas, the adrenal glands, and the ovaries and testes.

The secretion of hormones into the circulation is tightly controlled. Hormones are often secreted in diurnal, pulsatile, and cyclic patterns. Hormone secretion is regulated by perturbations in blood biochemistry, by other upstream-acting hormones, by neural impulses, and by negative feedback loops. Blood hormone concentrations are constantly monitored and adjusted to maintain optimal, steady-state levels. Once secreted, hormones act only on those target cells that express specific receptors.

Most disorders of the endocrine system are caused by either hyposecretion or hypersecretion of hormones. Hyposecretion often occurs when a hormone's gland of origin is damaged or otherwise impaired. Hypersecretion often results from the proliferation of tumors derived from hormone-secreting cells. Inappropriate hormone levels may also be caused by defects in regulatory feedback loops or in the processing of hormone precursors. Endocrine malfunction may also occur when the target cell fails to respond to the hormone.

Hormones can be classified biochemically as polypeptides, steroids, eicosanoids, or amines. Polypeptides, which include diverse hormones such as insulin and growth hormone, vary in size and function and are often synthesized as inactive precursors that are processed intracellularly into mature, active forms. Amines, which include epinephrine and dopamine, are amino acid derivatives that function in neuroendocrine signaling. Steroids, which include the cholesterol-derived hormones estrogen and testosterone, function in sexual development and reproduction. Eicosanoids, which include prostaglandins and prostacyclins, are fatty acid derivatives that function in a variety of processes. Most polypeptides and some amines are soluble in the circulation where they are highly susceptible to proteolytic degradation within seconds after their secretion. Steroids and lipids are insoluble and must be transported in the circulation by carrier proteins. The following discussion will focus primarily on polypeptide hormones.

Hormones secreted by the hypothalamus and pituitary gland play a critical role in endocrine function by coordinately regulating hormonal secretions from other endocrine glands in response to neural signals. Hypothalamic hormones include thyrotropin-releasing hormone, gonadotropin-releasing hormone, somatostatin, growth-hormone releasing factor, corticotropin-releasing hormone, substance P, dopamine, and prolactin-releasing hormone. These hormones directly regulate the secretion of hormones from the anterior lobe of the pituitary. Hormones secreted by the anterior pituitary include adrenocorticotropic hormone (ACTH), melanocyte-stimulating hormone, somatotropic hormones such as growth hormone and prolactin, glycoprotein hormones such as thyroid-stimulating hormone, luteinizing hormone (LH), and follicle-stimulating hormone (FSH), β-lipotropin, and β-endorphins. These hormones regulate hormonal secretions from the thyroid, pancreas, and adrenal glands, and act directly on the reproductive organs to stimulate ovulation and spermatogenesis. The posterior pituitary synthesizes and secretes antidiuretic hormone (ADH, vasopressin) and oxytocin.

Disorders of the hypothalamus and pituitary often result from lesions such as primary brain tumors, adenomas, infarction associated with pregnancy, hypophysectomy, aneurysms, vascular malformations, thrombosis, infections, immunological disorders, and complications due to head trauma. Such disorders have profound effects on the function of other endocrine glands. Disorders associated with hypopituitarism include hypogonadism, Sheehan syndrome, diabetes insipidus, Kallman's disease, Hand-Schuller-Christian disease, Letterer-Siwe disease, sarcoidosis, empty sella syndrome, and dwarfism. Disorders associated with hyperpituitarism include acromegaly, giantism, and syndrome of inappropriate ADH secretion (SIADH), often caused by benign adenomas.

Hormones secreted by the thyroid and parathyroid primarily control metabolic rates and the regulation of serum calcium levels, respectively. Thyroid hormones include calcitonin, somatostatin, and thyroid hormone. The parathyroid secretes parathyroid hormone. Disorders associated with hypothyroidism include goiter, myxedema, acute thyroiditis associated with bacterial infection, subacute thyroiditis associated with viral infection, autoimmune thyroiditis (Hashimoto's disease), and cretinism. Disorders associated with hyperthyroidism include thyrotoxicosis and its various forms, Grave's disease, pretibial myxedema, toxic multinodular goiter, thyroid carcinoma, and Plummer's disease. Disorders associated with hyperparathyroidism include Conn disease (chronic hypercalemia) leading to bone resorption and parathyroid hyperplasia.

Hormones secreted by the pancreas regulate blood glucose levels by modulating the rates of carbohydrate, fat, and protein metabolism. Pancreatic hormones include insulin, glucagon, amylin, γ-aminobutyric acid, gastrin, somatostatin, and pancreatic polypeptide. The principal disorder associated with pancreatic dysfunction is diabetes mellitus caused by insufficient insulin activity. Diabetes mellitus is generally classified as either Type I (insulin-dependent, juvenile diabetes) or Type II (non-insulin-dependent, adult diabetes). The treatment of both forms by insulin replacement therapy is well known. Diabetes mellitus often leads to acute complications such as hypoglycemia (insulin shock), coma, diabetic ketoacidosis, lactic acidosis, and chronic complications leading to disorders of the eye, kidney, skin, bone, joint, cardiovascular system, nervous system, and to decreased resistance to infection.

The anatomy, physiology, and diseases related to hormonal function are reviewed in McCance, K. L. and Huether, S. E. (1994) Pathophysiology: The Biological Basis for Disease in Adults and Children, Mosby-Year Book, Inc., St. Louis, Mo.; Greenspan, F. S. and Baxter, J. D. (1994) Basic and Clinical Endocrinology, Appleton and Lange, East Norwalk, Conn.

Growth Factors

Growth factors are secreted proteins that mediate intercellular communication. Unlike hormones, which travel great distances via the circulatory system, most growth factors are primarily local mediators that act on neighboring cells. Most growth factors contain a hydrophobic N-terminal signal peptide sequence which directs the growth factor into the secretory pathway. Most growth factors also undergo post-translational modifications within the secretory pathway. These modifications can include proteolysis, glycosylation, phosphorylation, and intramolecular disulfide bond formation. Once secreted, growth factors bind to specific receptors on the surfaces of neighboring target cells, and the bound receptors trigger intracellular signal transduction pathways. These signal transduction pathways elicit specific cellular responses in the target cells. These responses can include the modulation of gene expression and the stimulation or inhibition of cell division, cell differentiation, and cell motility.

Growth factors fall into at least two broad and overlapping classes. The broadest class includes the large polypeptide growth factors, which are wide-ranging in their effects. These factors include epidermal growth factor (EGF), fibroblast growth factor (FGF), transforming growth factor-β (TGF-β), insulin-like growth factor (IGF), nerve growth factor (NGF), and platelet-derived growth factor (PDGF), each defining a family of numerous related factors. The large polypeptide growth factors, with the exception of NGF, act as mitogens on diverse cell types to stimulate wound healing, bone synthesis and remodeling, extracellular matrix synthesis, and proliferation of epithelial, epidermal, and connective tissues. Members of the TGF-β, EGF, and FGF families also function as inductive signals in the differentiation of embryonic tissue. NGF functions specifically as a neurotrophic factor, promoting neuronal growth and differentiation.

EGF is a growth factor that stimulates proliferation of several epithelial tissues or cell lines. In addition to this mitogenic effect, EGF produces non-mitogenic effects in certain tissues. For example, in the stomach, EGF inhibits gastric acid secretion by parietal cells (Massagué, J. and Pandiella, A. (1993) Annu. Rev. Biochem. 62:515-541). EGF is produced as a larger precursor and contains an N-terminal signal peptide sequence that is thought to aid in localization of EGF to the plasma membrane. EGF contains three repeats of the calcium-binding EGF-like domain signature sequence. This signature sequence is about forty amino acid residues in length and includes six conserved cysteine residues, and a calcium-binding site near the N-terminus of the signature sequence. A number of proteins that contain calcium-binding EGF-like domain signature sequences are involved in growth and differentiation. Examples include bone morphogenic protein 1, which induces the formation of cartilage and bone; crumbs, which is a Drosophila melanogaster epithelial development protein; Notch and a number of its homologs, which are involved in neural growth and differentiation; and transforming growth factor beta-i binding protein (Expasy PROSITE document PDOC00913; Soler, C. and Carpenter, G., in Nicola, N. A. (1994) The Cytokine Facts Book, Oxford University Press, Oxford, UK, pp 193-197).

Another class of growth factors includes the hematopoietic growth factors, which are narrow in their target specificity. These factors stimulate the proliferation and differentiation of blood cells such as B-lymphocytes, T-lymphocytes, erythrocytes, platelets, eosinophils, basophils, neutrophils, macrophages, and their stem cell precursors. These factors include the colony-stimulating factors (G-CSF, M-CSF, GM-CSF, and CSF1-3), erythropoietin, and the cytokines. The cytokines are specialized hematopoietic factors secreted by cells of the immune system and are discussed in detail below.

Growth factors play critical roles in neoplastic transformation of cells in vitro and in tumor progression in vivo. Overexpression of the large polypeptide growth factors promotes the proliferation and transformation of cells in culture. Inappropriate expression of these growth factors by tumor cells in vivo may contribute to tumor vascularization and metastasis. Inappropriate activity of hematopoietic growth factors can result in anemias, leukemias, and lymphomas. Moreover, growth factors are both structurally and functionally related to oncoproteins, the potentially cancer-causing products of proto-oncogenes. Certain FGF and PDGF family members are themselves homologous to oncoproteins, whereas receptors for some members of the EGF, NGF, and FGF families are encoded by proto-oncogenes. Growth factors also affect the transcriptional regulation of both proto-oncogenes and oncosuppressor genes. (Pimentel, E. (1994) Handbook of Growth Factors, CRC Press, Ann Arbor, Mich.; McKay, I. and Leigh, I., eds. (1993) Growth Factors: A Practical Approach, Oxford University Press, New York, N.Y.; Habenicht, A., ed. (1990) Growth Factors, Differentiation Factors, and Cytokines, Springer-Verlag, New York, N.Y.)

In addition, some of the large polypeptide growth factors play crucial roles in the induction of the primordial germ layers in the developing embryo. This induction ultimately results in the formation of the embryonic mesoderm, ectoderm, and endoderm which in turn provide the framework for the entire adult body plan. Disruption of this inductive process would be catastrophic to embryonic development.

Small Peptide Factors—Neuropeptides and Vasomediators

Neuropeptides and vasomediators (NP/VM) comprise a family of small peptide factors, typically of 20 amino acids or less. These factors generally function in neuronal excitation and inhibition of vasoconstriction/vasodilation, muscle contraction, and hormonal secretions from the brain and other endocrine tissues. Included in this family are neuropeptides and neuropeptide hormones such as bombesin, neuropeptide Y, neurotensin, neuromedin N, melanocortins, opioids, galanin, somatostatin, tachykinins, urotensin II and related peptides involved in smooth muscle stimulation, vasopressin, vasoactive intestinal peptide, and circulatory system-borne signaling molecules such as angiotensin, complement, calcitonin, endothelins, formyl-methionyl peptides, glucagon, cholecystokinin, gastrin, and many of the peptide hormones discussed above. NP/VMs can transduce signals directly, modulate the activity or release of other neurotransmitters and hormones, and act as catalytic enzymes in signaling cascades. The effects of NP/VMs range from extremely brief to long-lasting. (Reviewed in Martin, C. R. et al. (1985) Endocrine Physiology, Oxford University Press, New York, N.Y., pp. 57-62.)

The FMRFamide-like neuropeptides are a class of peptides found particularly in the brain, spinal cord, and gastrointestinal tract. FMRFamide-related peptides interact with opiate receptors (Raffa, R. B. (1991) NIDA Res. Monogr. 105:243-249).

Bombesin is a neuropeptide involved in appetite and stress response. Bombesin-like peptides are released at the central nucleus of the amygdala in response to both stress and food intake (Merali, Z. et al. (1998) J. Neurosci. 18:4758-4766). Bombesin has been shown to decrease food intake, increase the duration of slow wave sleep, and increase the concentration of both blood glucose and glucagon (Even, P. C. et al. (1991) Physiol. Behav. 49:439-442).

Cytokines

Cytokines comprise a family of signaling molecules that modulate the immune system and the inflammatory response. Cytokines are usually secreted by leukocytes, or white blood cells, in response to injury or infection. Cytokines function as growth and differentiation factors that act primarily on cells of the immune system such as B- and T-lymphocytes, monocytes, macrophages, and granulocytes. Like other signaling molecules, cytokines bind to specific plasma membrane receptors and trigger intracellular signal transduction pathways which alter gene expression patterns. There is considerable potential for the use of cytokines in the treatment of inflammation and immune system disorders.

Cytokine structure and function have been extensively characterized in vitro. Most cytokines are small polypeptides of about 30 kilodaltons or less. Over 50 cytokines have been identified from human and rodent sources. Examples of cytokine subfamilies include the interferons (IFN-α, -β, and -γ), the interleukins (IL1-IL13), the tumor necrosis factors (TNF-α and -β), and the chemokines. Many cytokines have been produced using recombinant DNA techniques, and the activities of individual cytokines have been determined in vitro. These activities include regulation of leukocyte proliferation, differentiation, and motility.

The activity of an individual cytokine in vitro may not reflect the full scope of that cytokine's activity in vivo. Cytokines are not expressed individually in vivo but are instead expressed in combination with a multitude of other cytokines when the organism is challenged with a stimulus. Together, these cytokines collectively modulate the immune response in a manner appropriate for that particular stimulus. Therefore, the physiological activity of a cytokine is determined by the stimulus itself and by complex interactive networks among co-expressed cytokines which may demonstrate both synergistic and antagonistic relationships.

Recently, a unique cytokine has been characterized with a likely role in regulating fibrogenesis associated with cases of chronic inflammation. This cytokine, fibrosin, has no obvious homology with other proteins in the GenBank database. A 36-amino acid synthetic peptide constructed from the deduced amino acid sequence of human fibrosin stimulates fibroblast growth at subnanomolar concentrations. Tissue fibrosis is a serious complication that accompanies chronic inflammation. A number of fibrogenic cytokines act in concert to stimulate the growth of fiborblasts and the extracellular matrix components associated with fibrosis. (Prakash, S. and P. W. Robbins (1998) DNA Cell Bio. 17:879-884).

Interleukin-10 (IL-10) is one of the better studied cytokines. In humans IL-10 is a secreted 18 kilodalton protein produced by some T and B lymphocytes and macrophages. There are four cysteine residues in the IL-10 protein that are conserved in human, murine and viral IL-10. Two of these cysteines are involved in the formation of intramolecular disulfide bonds. IL-10 can inhibit cytokine production by T cells, inhibit cytokine synthesis by macrophages, and stimulate proliferation of thymocytes, T cells and B cells in addition to megakaryocytes, and other haemopoietic cells. (Nicola, N. A. (1994) Guidebook to Cytokines and Their Receptors Oxford University Press, New York, N.Y., pp. 84-85).

Low homologies between various cytokine family members make it difficult to establish relationships between known members and newly discovered cytokines. Homologies within families can be 25% or lower, and conserved amino acids may be clustered in small domains or repeats. Often only a seeming chance similarity exits between family members until structural information clarifies homologies. Conserved disulfide bridges are a strong indicator of conserved or similar protein structure and or folding. For example, IL-10 molecules from several sources share four conserved cysteines that participate in structure determining intramolecular contacts. (Callard, R. and A. Gearing. (1994) In The Cytokine Factsbook, Academic Press, San Diego Calif., p. 18).

Chemokines comprise a cytokine subfamily with over 30 members. (Reviewed in Wells, T. N. C. and Peitsch, M. C. (1997) J. Leukoc. Biol. 61:545-550.) Chemokines were initially identified as chemotactic proteins that recruit monocytes and macrophages to sites of inflammation. Recent evidence indicates that chemokines may also play key roles in hematopoiesis and HIV-1 infection. Chemokines are small proteins which range from about 6-15 kilodaltons in molecular weight. Chemokines are further classified as C, CC, CXC, or CX₃C based on the number and position of critical cysteine residues. The CC chemokines, for example, each contain a conserved motif consisting of two consecutive cysteines followed by two additional cysteines which occur downstream at 24- and 16-residue intervals, respectively (ExPASy PROSITE database, documents PS00472 and PDOC00434). The presence and spacing of these four cysteine residues are highly conserved, whereas the intervening residues diverge significantly. However, a conserved tyrosine located about 15 residues downstream of the cysteine doublet seems to be important for chemotactic activity. Most of the human genes encoding CC chemokines are clustered on chromosome 17, although there are a few examples of CC chemokine genes that map elsewhere. Other chemokines include lymphotactin (C chemokine); macrophage chemotactic and activating factor (MCAF/MCP-1; CC chemokine); platelet factor 4 and IL-8 (CXC chemokines); and fractalkine and neurotractin (CX₃C chemokines). (Reviewed in Luster, A. D. (1998) N. Engl. J. Med. 338:436-445.)

The discovery of new extracellular signaling molecules and the polynucleotides encoding them satisfies a need in the art by providing new compositions which are useful in the diagnosis, prevention, and treatment of infections and gastrointestinal, neurological, reproductive, autoimmune/inflammatory, and cell proliferative disorders including cancer.

### SUMMARY OF THE INVENTION

The invention features purified polypeptides, extracellular signaling molecules, referred to collectively as “EXCS” and individually as “EXCS-1,” “EXCS-2,” “EXCS-3,” “EXCS-4,” “EXCS-5,” “EXCS-6,” “EXCS-7,” “EXCS-8,” “EXCS-9,” “EXCS-10,” “EXCS-11,” “EXCS-12,” “EXCS-13,” “EXCS-14,” “EXCS-15,” “EXCS-16,” “EXCS-17,” “EXCS-18,” “EXCS- 19,” “EXCS-20,” “EXCS-21,” “EXCS-22,” “EXCS-23,” “EXCS-24,” “EXCS-25,” and “EXCS-26.” In one aspect, the invention provides an isolated polypeptide comprising a) an amino acid sequence selected from the group consisting of SEQ ID NO: 1-26, b) a naturally occurring amino acid sequence having at least 90% sequence identity to an amino acid sequence selected from the group consisting of SEQ ID NO: 1-26, c) a biologically active fragment of an amino acid sequence selected from the group consisting of SEQ ID NO: 1-26, or d) an immunogenic fragment of an amino acid sequence selected from the group consisting of SEQ ID NO: 1-26. In one alternative, the invention provides an isolated polypeptide comprising the amino acid sequence of SEQ ID NO: 1-26.

The invention further provides an isolated polynucleotide encoding a polypeptide comprising a) an amino acid sequence selected from the group consisting of SEQ ID NO: 1-26, b) a naturally occurring amino acid sequence having at least 90% sequence identity to an amino acid sequence selected from the group consisting of SEQ ID NO: 1-26, c) a biologically active fragment of an amino acid sequence selected from the group consisting of SEQ ID NO: 1-26, or d) an immunogenic fragment of an amino acid sequence selected from the group consisting of SEQ ID NO: 1-26. In one alternative, the polynucleotide is selected from the group consisting of SEQ ID NO:27-52.

Additionally, the invention provides a recombinant polynucleotide comprising a promoter sequence operably linked to a polynucleotide encoding a polypeptide comprising a) an amino acid sequence selected from the group consisting of SEQ ID NO: 1-26, b) a naturally occurring amino acid sequence having at least 90% sequence identity to an amino acid sequence selected from the group consisting of SEQ ID NO: 1-26, c) a biologically active fragment of an amino acid sequence selected from the group consisting of SEQ ID NO: 1-26, or d) an immunogenic fragment of an amino acid sequence selected from the group consisting of SEQ ID NO: 1-26. In one alternative, the invention provides a cell transformed with the recombinant polynucleotide. In another alternative, the invention provides a transgenic organism comprising the recombinant polynucleotide.

The invention also provides a method for producing a polypeptide comprising a) an amino acid sequence selected from the group consisting of SEQ ID NO: 1-26, b) a naturally occurring amino acid sequence having at least 90% sequence identity to an amino acid sequence selected from the group consisting of SEQ ID NO: 1-26, c) a biologically active fragment of an amino acid sequence selected from the group consisting of SEQ ID NO: 1-26, or d) an immunogenic fragment of an amino acid sequence selected from the group consisting of SEQ ID NO: 1-26. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Additionally, the invention provides an isolated antibody which specifically binds to a polypeptide comprising a) an amino acid sequence selected from the group consisting of SEQ ID NO: 1-26, b) a naturally occurring amino acid sequence having at least 90% sequence identity to an amino acid sequence selected from the group consisting of SEQ ID NO: 1-26, c) a biologically active fragment of an amino acid sequence selected from the group consisting of SEQ ID NO: 1-26, or d) an immunogenic fragment of an amino acid sequence selected from the group consisting of SEQ ID NO: 1-26.

The invention further provides an isolated polynucleotide comprising a) a polynucleotide sequence selected from the group consisting of SEQ ID NO:27-52, b) a naturally occurring polynucleotide sequence having at least 90% sequence identity to a polynucleotide sequence selected from the group consisting of SEQ ID NO:27-52, c) a polynucleotide sequence complementary to a), or d) a polynucleotide sequence complementary to b). In one alternative, the polynucleotide comprises at least 60 contiguous nucleotides.

Additionally, the invention provides a method for detecting a target polynucleotide in a sample, said target polynucleotide having a sequence of a polynucleotide comprising a) a polynucleotide sequence selected from the group consisting of SEQ ID NO:27-52, b) a naturally occurring polynucleotide sequence having at least 90% sequence identity to a polynucleotide sequence selected from the group consisting of SEQ ID NO:27-52, c) a polynucleotide sequence complementary to a), or d) a polynucleotide sequence complementary to b). The method comprises a) hybridizing the sample with a probe comprising at least 16 contiguous nucleotides comprising a sequence complementary to said target polynucleotide in the sample, and which probe specifically hybridizes to said target polynucleotide, under conditions whereby a hybridization complex is formed between said probe and said target polynucleotide, and b) detecting the presence or absence of said hybridization complex, and optionally, if present, the amount thereof. In one alternative, the probe comprises at least 30 contiguous nucleotides. In another alternative, the probe comprises at least 60 contiguous nucleotides.

The invention further provides a pharmaceutical composition comprising an effective amount of a polypeptide comprising a) an amino acid sequence selected from the group consisting of SEQ ID NO: 1-26, b) a naturally occurring amino acid sequence having at least 90% sequence identity to an amino acid sequence selected from the group consisting of SEQ ID NO: 1-26, c) a biologically active fragment of an amino acid sequence selected from the group consisting of SEQ ID NO: 1-26, or d) an immunogenic fragment of an amino acid sequence selected from the group consisting of SEQ ID NO: 1-26, and a pharmaceutically acceptable excipient. The invention additionally provides a method of treating a disease or condition associated with decreased expression of functional EXCS, comprising administering to a patient in need of such treatment the pharmaceutical composition.

The invention also provides a method for screening a compound for effectiveness as an agonist of a polypeptide comprising a) an amino acid sequence selected from the group consisting of SEQ ID NO: 1-26, b) a naturally occurring amino acid sequence having at least 90% sequence identity to an amino acid sequence selected from the group consisting of SEQ ID NO: 1-26, c) a biologically active fragment of an amino acid sequence selected from the group consisting of SEQ ID NO: 1-26, or d) an immunogenic fragment of an amino acid sequence selected from the group consisting of SEQ ID NO: 1-26. The method comprises a) exposing a sample comprising the polypeptide to a compound, and b) detecting agonist activity in the sample. In one alternative, the invention provides a pharmaceutical composition comprising an agonist compound identified by the method and a pharmaceutically acceptable excipient. In another alternative, the invention provides a method of treating a disease or condition associated with decreased expression of functional EXCS, comprising administering to a patient in need of such treatment the pharmaceutical composition.

Additionally, the invention provides a method for screening a compound for effectiveness as an antagonist of a polypeptide comprising a) an amino acid sequence selected from the group consisting of SEQ ID NO: 1-26, b) a naturally occurring amino acid sequence having at least 90% sequence identity to an amino acid sequence selected from the group consisting of SEQ ID NO: 1-26, c) a biologically active fragment of an amino acid sequence selected from the group consisting of SEQ ID NO: 1-26, or d) an immunogenic fragment of an amino acid sequence selected from the group consisting of SEQ ID NO: 1-26. The method comprises a) exposing a sample comprising the polypeptide to a compound, and b) detecting antagonist activity in the sample. In one alternative, the invention provides a pharmaceutical composition comprising an antagonist compound identified by the method and a pharmaceutically acceptable excipient. In another alternative, the invention provides a method of treating a disease or condition associated with overexpression of functional EXCS, comprising administering to a patient in need of such treatment the pharmaceutical composition.

The invention further provides a method for screening a compound for effectiveness in altering expression of a target polynucleotide, wherein said target polynucleotide comprises a sequence selected from the group consisting of SEQ ID NO:27-52, the method comprising a) exposing a sample comprising the target polynucleotide to a compound, and b) detecting altered expression of the target polynucleotide.

### BRIEF DESCRIPTION OF THE TABLES AND FIGURE

Table 1 shows polypeptide and nucleotide sequence identification numbers (SEQ ID NOs), clone identification numbers (clone IDs), cDNA libraries, and cDNA fragments used to assemble full-length sequences encoding EXCS.

Table 2 shows features of each polypeptide sequence, including potential motifs, homologous sequences, and methods, algorithms, and searchable databases used for analysis of EXCS.

Table 3 shows selected fragments of each nucleic acid sequence; the tissue-specific expression patterns of each nucleic acid sequence as determined by northern analysis; diseases, disorders, or conditions associated with these tissues; and the vector into which each cDNA was cloned.

Table 4 describes the tissues used to construct the cDNA libraries from which cDNA clones encoding EXCS were isolated.

Table 5 shows the tools, programs, and algorithms used to analyze EXCS, along with applicable descriptions, references, and threshold parameters.

FIGS. 1A and 1B show the amino acid sequence alignment among EXCS-18 (SEQ ID NO:18), interleukin-10 (GI 511295), IL-10 precursor (GI 1841298) and interleukin-10 precursor-human (GI 106805), produced using the multisequence alignment program of LASERGENE software (DNASTAR, Madison Wis.).

### DESCRIPTION OF THE INVENTION

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

### DEFINITIONS

“EXCS” refers to the amino acid sequences of substantially purified EXCS obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of EXCS. Agonists may include proteins, nucleic acids, carbohydrates, small molecules, or any other compound or composition which modulates the activity of EXCS either by directly interacting with EXCS or by acting on components of the biological pathway in which EXCS participates.

An “allelic variant” is an alternative form of the gene encoding EXCS. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding EXCS include those sequences with deletions, insertions, or substitutions of different nucleotides, resulting in a polypeptide the same as EXCS or a polypeptide with at least one functional characteristic of EXCS. Included within this definition are polymorphisms which may or may not be readily detectable using a particular oligonucleotide probe of the polynucleotide encoding EXCS, and improper or unexpected hybridization to allelic variants, with a locus other than the normal chromosomal locus for the polynucleotide sequence encoding EXCS. The encoded protein may also be “altered,” and may contain deletions, insertions, or substitutions of amino acid residues which produce a silent change and result in a functionally equivalent EXCS. Deliberate amino acid substitutions may be made on the basis of similarity in polarity, charge, solubility, hydrophobicity, hydrophilicity, and/or the amphipathic nature of the residues, as long as the biological or immunological activity of EXCS is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to an amino acid sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies well known in the art.

The term “antagonist” refers to a molecule which inhibits or attenuates the biological activity of EXCS. Antagonists may include proteins such as antibodies, nucleic acids, carbohydrates, small molecules, or any other compound or composition which modulates the activity of EXCS either by directly interacting with EXCS or by acting on components of the biological pathway in which EXCS participates.

The term “antibody” refers to intact immunoglobulin molecules as well as to fragments thereof, such as Fab, F(ab′)₂, and Fv fragments, which are capable of binding an epitopic determinant. Antibodies that bind EXCS polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “antisense” refers to any composition capable of base-pairing with the “sense” strand of a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” refers to the capability of the natural, recombinant, or synthetic EXCS, or of any oligopeptide thereof, to induce a specific immune response in appropriate animals or cells and to bind with specific antibodies.

The terms “complementary” and “complementarity” refer to the natural binding of polynucleotides by base pairing. For example, the sequence “5′A-G-T 3′” bonds to the complementary sequence “3′T-C-A 5′.” Complementarity between two single-stranded molecules may be “partial,” such that only some of the nucleic acids bind, or it may be “complete,” such that total complementarity exists between the single stranded molecules. The degree of complementarity between nucleic acid strands has significant effects on the efficiency and strength of the hybridization between the nucleic acid strands. This is of particular importance in amplification reactions, which depend upon binding between nucleic acid strands, and in the design and use of peptide nucleic acid (PNA) molecules.

A “composition comprising a given polynucleotide sequence” and a “composition comprising a given amino acid sequence” refer broadly to any composition containing the given polynucleotide or amino acid sequence. The composition may comprise a dry formulation or an aqueous solution. Compositions comprising polynucleotide sequences encoding EXCS or fragments of EXCS may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been resequenced to resolve uncalled bases, extended using the XL-PCR kit (Perkin-Elmer, Norwalk Conn.) in the 5′ and/or the 3′ direction, and resequenced, or which has been assembled from the overlapping sequences of one or more Incyte Clones and, in some cases, one or more public domain ESTs, using a computer program for fragment assembly, such as the GELVIEW fragment assembly system (GCG, Madison Wis.). Some sequences have been both extended and assembled to produce the consensus sequence.

“Conservative amino acid substitutions” are those substitutions that, when made, least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to the chemical modification of a polypeptide sequence, or a polynucleotide sequence. Chemical modifications of a polynucleotide sequence can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “fragment” is a unique portion of EXCS or the polynucleotide encoding EXCS which is identical in sequence to but shorter in length than the parent sequence. A fragment may comprise up to the entire length of the defined sequence, minus one nucleotide/amino acid residue. For example, a fragment may comprise from 5 to 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50% of a polypeptide) as shown in a certain defined sequence. Clearly these lengths are exemplary, and any length that is supported by the specification, including the Sequence Listing, tables, and figures, may be encompassed by the present embodiments.

A fragment of SEQ ID NO:27-52 comprises a region of unique polynucleotide sequence that specifically identifies SEQ ID NO:27-52, for example, as distinct from any other sequence in the same genome. A fragment of SEQ ID NO:27-52 is useful, for example, in hybridization and amplification technologies and in analogous methods that distinguish SEQ ID NO: 27-52 from related polynucleotide sequences. The precise length of a fragment of SEQ ID NO:27-52 and the region of SEQ ID NO:27-52 to which the fragment corresponds are routinely determinable by one of ordinary skill in the art based on the intended purpose for the fragment.

A fragment of SEQ ID NO: 1-26 is encoded by a fragment of SEQ ID NO:27-52. A fragment of SEQ ID NO: 1-26 comprises a region of unique amino acid sequence that specifically identifies SEQ ID NO: 1-26. For example, a fragment of SEQ ID NO: 1-26 is useful as an immunogenic peptide for the development of antibodies that specifically recognize SEQ ID NO: 1-26. The precise length of a fragment of SEQ ID NO: 1-26 and the region of SEQ ID NO: 1-26 to which the fragment corresponds are routinely determinable by one of ordinary skill in the art based on the intended purpose for the fragment.

The term “similarity” refers to a degree of complementarity. There may be partial similarity or complete similarity. The word “identity” may substitute for the word “similarity.” A partially complementary sequence that at least partially inhibits an identical sequence from hybridizing to a target nucleic acid is referred to as “substantially similar.” The inhibition of hybridization of the completely complementary sequence to the target sequence may be examined using a hybridization assay (Southern or northern blot, solution hybridization, and the like) under conditions of reduced stringency. A substantially similar sequence or hybridization probe will compete for and inhibit the binding of a completely similar (identical) sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% similarity or identity). In the absence of non-specific binding, the substantially similar sequence or probe will not hybridize to the second non-complementary target sequence.

The phrases “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequence pairs.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.9 (May-07-1999) set at default parameters. Such default parameters may be, for example:

Matrix: BLOSUM62

Reward for match: 1

Penalty for mismatch: −2

Open Gap: 5 and Extension Gap: 2 penalties

Gap×drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the hydrophobicity and acidity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.9 (May 7, 1999) with blastp set at default parameters. Such default parameters may be, for example:

Matrix: BLOSUM62

Open Gap: 11 and Extension Gap: 1 penalties

Gap×drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size, and which contain all of the elements required for stable mitotic chromosome segregation and maintenance.

The term “humanized antibody” refers to antibody molecules in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of identity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml denatured salmon sperm DNA.

Generally, stringency of hybridization is expressed, in part, with reference to the temperature under which the wash step is carried out. Generally, such wash temperatures are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook et al., 1989, Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of EXCS which is capable of eliciting an immune response when introduced into a living organism, for example, a mammal. The term “immunogenic fragment” also includes any polypeptide or oligopeptide fragment of EXCS which is useful in any of the antibody production methods disclosed herein or known in the art.

The term “microarray” refers to an arrangement of distinct polynucleotides on a substrate.

The terms “element” and “array element” in a microarray context, refer to hybridizable polynucleotides arranged on the surface of a substrate.

The term “modulate” refers to a change in the activity of EXCS. For example, modulation may cause an increase or a decrease in protein activity, binding characteristics, or any other biological, functional, or immunological properties of EXCS.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with the second nucleic acid sequence. For instance, a promoter is operably linked to a coding sequence if the promoter affects the transcription or expression of the coding sequence. Generally, operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Probe” refers to nucleic acid sequences encoding EXCS, their complements, or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook et al., 1989, Molecular Cloning: A Laboratory Manual, 2^{nd} ed., vol. 1-3, Cold Spring Harbor Press, Plainview N.Y.; Ausubel et al., 1987, Current Protocols in Molecular Biology, Greene Publ. Assoc. & Wiley-Intersciences, New York N.Y.; Innis et al., 1990, PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

The term “sample” is used in its broadest sense. A sample suspected of containing nucleic acids encoding EXCS, or fragments thereof, or EXCS itself, may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide containing the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least 60% free, preferably at least 75% free, and most preferably at least 90% free from other components with which they are naturally associated.

A “substitution” refers to the replacement of one or more amino acids or nucleotides by different amino acids or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

“Transformation” describes a process by which exogenous DNA enters and changes a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, viral infection, electroporation, heat shock, lipofection, and particle bombardment. The term “transformed” cells includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, and plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 95% or at least 98% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 95%, or at least 98% or greater sequence identity over a certain defined length of one of the polypeptides.

### THE INVENTION

The invention is based on the discovery of new human extracellular signaling molecules (EXCS), the polynucleotides encoding EXCS, and the use of these compositions for the diagnosis, treatment, or prevention of infections and gastrointestinal, neurological, reproductive, autoimmune/inflammatory, and cell proliferative disorders including cancer.

Table 1 lists the Incyte clones used to assemble full length nucleotide sequences encoding EXCS. Columns 1 and 2 show the sequence identification numbers (SEQ ID NOs) of the polypeptide and nucleotide sequences, respectively. Column 3 shows the clone IDs of the Incyte clones in which nucleic acids encoding each EXCS were identified, and column 4 shows the cDNA libraries from which these clones were isolated. Column 5 shows Incyte clones and their corresponding cDNA libraries. Clones for which cDNA libraries are not indicated were derived from pooled cDNA libraries. In some cases, GenBank sequence identifiers are also shown in column 5. The Incyte clones and GenBank cDNA sequences, where indicated, in column 5 were used to assemble the consensus nucleotide sequence of each EXCS and are useful as fragments in hybridization technologies.

The columns of Table 2 show various properties of each of the polypeptides of the invention: column 1 references the SEQ ID NO; column 2 shows the number of amino acid residues in each polypeptide; column 3 shows potential phosphorylation sites; column 4 shows potential glycosylation sites; column 5 shows the amino acid residues comprising signature sequences and motifs; column 6 shows homologous sequences as identified by BLAST analysis along with relevant citations, all of which are expressly incorporated by reference herein in their entirety; and column 7 shows analytical methods and in some cases, searchable databases to which the analytical methods were applied. The methods of column 7 were used to characterize each polypeptide through sequence homology and protein motifs. Of particular note is the presence of one or more cysteine residues in each of the polypeptide sequences of SEQ ID NO: 1-10.

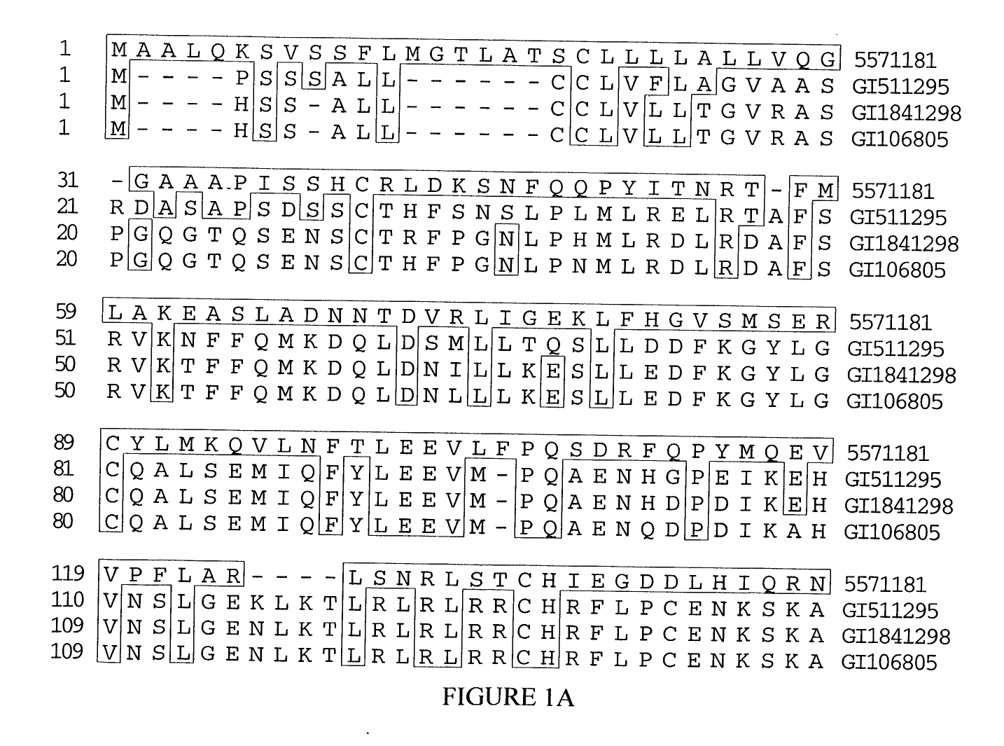

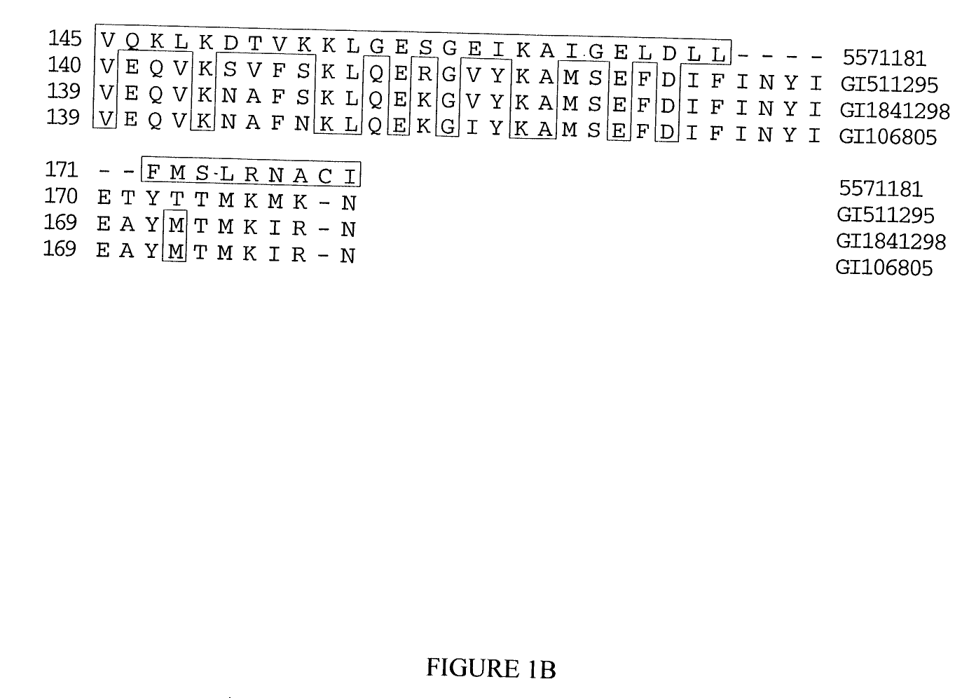

FIGS. 1A, and 1B show the amino acid sequence alignment among EXCS-18 (SEQ ID NO:18), interleukin-10 (GI 511295; SEQ ID NO:53), interleukin-10 precursor (GI 1841298; SEQ ID NO:54) and interleukin-10 precursor-human (GI 106805; SEQ ID NO:55) with conserved amino acid residues boxed. The alignments illustrate an overall protein length in the range of 178-179 residues for all four proteins, indicating that SEQ ID NO: 18 shares structural similarity with GI 511295, GI 1841298, and GI 106805 on the basis of molecule length. It is also noteworthy that SEQ ID NO: 18 shares four out of six highly conserved cysteine residues found in GI 511295, GI 1841298, and GI 106805 at positions C20, C40, C89 and C132. Furthermore, three of these cysteines (C40, C89 and C132) are known to be directly involved in intramolecular disulfide bridge formation within IL-10 molecules, thus illustrating homology and possible secondary structural similarity of SEQ ID NO: 18 to GI 511295, GI 1841298, and GI 106805. Additional homology of SEQ ID NO: 18 to GI 511295, GI 1841298, and GI 106805 is apparent as numerous conserved amino acid residues, including a number of basic and acidic residues, and in particular, two structurally relevant proline residues at positions 106 and 113.

The columns of Table 3 show the tissue-specificity and diseases, disorders, or conditions associated with nucleotide sequences encoding EXCS. The first column of Table 3 lists the nucleotide SEQ ID NOs. Column 2 lists fragments of the nucleotide sequences of column 1. These fragments are useful, for example, in hybridization or amplification technologies to identify SEQ ID NO:27-52 and to distinguish between SEQ ID NO:27-52 and related polynucleotide sequences. The polypeptides encoded by these fragments are useful, for example, as immunogenic peptides. Column 3 lists tissue categories which express EXCS as a fraction of total tissues expressing EXCS. Column 4 lists diseases, disorders, or conditions associated with those tissues expressing EXCS as a fraction of total tissues expressing EXCS. Of particular note is the expression of SEQ ID NO:30. This sequence is detected in six cDNA libraries, all of which were constructed independently using RNA isolated from prostate tissue. Therefore, SEQ ID NO:30 is useful, for example, as a prostate-specific marker for tissue-typing and for diagnosis of diseases of the prostate. SEQ ID NO:43 is specifically expressed in islet cells and in islet cell tumor only. Of particular note is the expression of SEQ ID NO:45 exclusively in hematopoietic/immune tissues. Column 5 lists the vectors used to subclone each cDNA library.

The columns of Table 4 show descriptions of the tissues used to construct the cDNA libraries from which cDNA clones encoding EXCS were isolated. Column 1 references the nucleotide SEQ ID NOs, column 2 shows the cDNA libraries from which these clones were isolated, and column 3 shows the tissue origins and other descriptive information relevant to the cDNA libraries in column 2.

SEQ ID NO:47 maps to chromosome 2 within the interval from 77.1 to 84.0 centiMorgans. This interval also contains a gene associated with stimulation of DNA synthesis.

The invention also encompasses EXCS variants. A preferred EXCS variant is one which has at least about 80%, or alternatively at least about 90%, or even at least about 95% amino acid sequence identity to the EXCS amino acid sequence, and which contains at least one functional or structural characteristic of EXCS.

The invention also encompasses polynucleotides which encode EXCS. In a particular embodiment, the invention encompasses a polynucleotide sequence comprising a sequence selected from the group consisting of SEQ ID NO:27-52, which encodes EXCS. The polynucleotide sequences of SEQ ID NO:27-52, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses a variant of a polynucleotide sequence encoding EXCS. In particular, such a variant polynucleotide sequence will have at least about 70%, or alternatively at least about 85%, or even at least about 95% polynucleotide sequence identity to the polynucleotide sequence encoding EXCS. A particular aspect of the invention encompasses a variant of a polynucleotide sequence comprising a sequence selected from the group consisting of SEQ ID NO:27-52 which has at least about 70%, or alternatively at least about 85%, or even at least about 95% polynucleotide sequence identity to a nucleic acid sequence selected from the group consisting of SEQ ID NO:27-52. Any one of the polynucleotide variants described above can encode an amino acid sequence which contains at least one functional or structural characteristic of EXCS.

It will be appreciated by those skilled in the art that as a result of the degeneracy of the genetic code, a multitude of polynucleotide sequences encoding EXCS, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring EXCS, and all such variations are to be considered as being specifically disclosed.

Although nucleotide sequences which encode EXCS and its variants are generally capable of hybridizing to the nucleotide sequence of the naturally occurring EXCS under appropriately selected conditions of stringency, it may be advantageous to produce nucleotide sequences encoding EXCS or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding EXCS and its derivatives without altering the encoded amino acid sequences include the production of RNA transcripts having more desirable properties, such as a greater half-life, than transcripts produced from the naturally occurring sequence.

The invention also encompasses production of DNA sequences which encode EXCS and EXCS derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding EXCS or any fragment thereof.

Also encompassed by the invention are polynucleotide sequences that are capable of hybridizing to the claimed polynucleotide sequences, and, in particular, to those shown in SEQ ID NO:27-52 and fragments thereof under various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland OH), Taq polymerase (Perkin-Elmer), thermostable T7 polymerase (Amersham Pharmacia Biotech, Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Perkin-Elmer). Sequencing is then carried out using either the ABI 373 or 377 DNA sequencing system (Perkin-Elmer), the MEGABACE 1000 DNA sequencing system (Molecular Dynamics, Sunnyvale Calif.), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acid sequences encoding EXCS may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:81-86.) A third method, capture PCR, involves PCR amplification of DNA fragments adjacent to known sequences in human and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 Primer Analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full-length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Perkin-Elmer), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode EXCS may be cloned in recombinant DNA molecules that direct expression of EXCS, or fragments or functional equivalents thereof, in appropriate host cells. Due to the inherent degeneracy of the genetic code, other DNA sequences which encode substantially the same or a functionally equivalent amino acid sequence may be produced and used to express EXCS.

The nucleotide sequences of the present invention can be engineered using methods generally known in the art in order to alter EXCS-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of EXCS, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, sequences encoding EXCS may be synthesized, in whole or in part, using chemical methods well known in the art. (See, e.g., Caruthers, M. H. et al. (1980) Nucleic Acids Symp. Ser. 7:215-223; and Horn, T. et al. (1980) Nucleic Acids Symp. Ser. 7:225-232.) Alternatively, EXCS itself or a fragment thereof may be synthesized using chemical methods. For example, peptide synthesis can be performed using various solid-phase techniques. (See, e.g., Roberge, J. Y. et al. (1995) Science 269:202-204.) Automated synthesis may be achieved using the ABI 431A peptide synthesizer (Perkin-Elmer). Additionally, the amino acid sequence of EXCS, or any part thereof, may be altered during direct synthesis and/or combined with sequences from other proteins, or any part thereof, to produce a variant polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, T. (1984) Proteins, Structures and Molecular Properties, WH Freeman, New York N.Y.)

In order to express a biologically active EXCS, the nucleotide sequences encoding EXCS or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding EXCS. Such elements may vary in their strength and specificity. Specific initiation signals may also be used to achieve more efficient translation of sequences encoding EXCS. Such signals include the ATG initiation codon and adjacent sequences, e.g. the Kozak sequence. In cases where sequences encoding EXCS and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.)

Methods which are well known to those skilled in the art may be used to construct expression vectors containing sequences encoding EXCS and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16.)

A variety of expression vector/host systems may be utilized to contain and express sequences encoding EXCS. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. The invention is not limited by the host cell employed.

In bacterial systems, a number of cloning and expression vectors may be selected depending upon the use intended for polynucleotide sequences encoding EXCS. For example, routine cloning, subcloning, and propagation of polynucleotide sequences encoding EXCS can be achieved using a multifunctional E. coli vector such as PBLUESCRIPT (Stratagene, La Jolla Calif.) or PSPORT 1 plasmid (Life Technologies). Ligation of sequences encoding EXCS into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of EXCS are needed, e.g. for the production of antibodies, vectors which direct high level expression of EXCS may be used. For example, vectors containing the strong, inducible T5 or T7 bacteriophage promoter may be used.

Yeast expression systems may be used for production of EXCS. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of EXCS. Transcription of sequences encoding EXCS may be driven viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, sequences encoding EXCS may be ligated into an adenovirus transcription/translation complex consisting of the late promoter and tripartite leader sequence. Insertion in a non-essential E1 or E3 region of the viral genome may be used to obtain infective virus which expresses EXCS in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355.)

For long term production of recombinant proteins in mammalian systems, stable expression of EXCS in cell lines is preferred. For example, sequences encoding EXCS can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G418; and als and pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e.g., Hartman, S. C. and R. C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding EXCS is inserted within a marker gene sequence, transformed cells containing sequences encoding EXCS can be identified by the absence of marker gene function. Alternatively, a marker gene can be placed in tandem with a sequence encoding EXCS under the control of a single promoter. Expression of the marker gene in response to induction or selection usually indicates expression of the tandem gene as well.

In general, host cells that contain the nucleic acid sequence encoding EXCS and that express EXCS may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of EXCS using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on EXCS is preferred, but a competitive binding assay may be employed. These and other assays are well known in the art. (See, e.g., Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding EXCS include oligolabeling, nick translation, end-labeling, or PCR amplification using a labeled nucleotide. Alternatively, the sequences encoding EXCS, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding EXCS may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode EXCS may be designed to contain signal sequences which direct secretion of EXCS through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant nucleic acid sequences encoding EXCS may be ligated to a heterologous sequence resulting in translation of a fusion protein in any of the aforementioned host systems. For example, a chimeric EXCS protein containing a heterologous moiety that can be recognized by a commercially available antibody may facilitate the screening of peptide libraries for inhibitors of EXCS activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the EXCS encoding sequence and the heterologous protein sequence, so that EXCS may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In a further embodiment of the invention, synthesis of radiolabeled EXCS may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

Fragments of EXCS may be produced not only by recombinant means, but also by direct peptide synthesis using solid-phase techniques. (See, e.g., Creighton, supra, pp. 55-60.) Protein synthesis may be performed by manual techniques or by automation. Automated synthesis may be achieved, for example, using the ABI 431A peptide synthesizer (Perkin-Elmer). Various fragments of EXCS may be synthesized separately and then combined to produce the full length molecule.

### THERAPEUTICS

Chemical and structural similarity, e.g., in the context of sequences and motifs, exists between regions of EXCS and extracellular signaling molecules. In addition, the expression of EXCS is closely associated with reproductive, cardiovascular, nervous, gastrointestinal, cancerous, hematopoietic/immune, cell proliferative and inflamed tissue. Therefore, EXCS appears to play a role in infections and gastrointestinal, neurological, reproductive, autoimmune/inflammatory, and cell proliferative disorders including cancer. In the treatment of disorders associated with increased EXCS expression or activity, it is desirable to decrease the expression or activity of EXCS. In the treatment of disorders associated with decreased EXCS expression or activity, it is desirable to increase the expression or activity of EXCS.

Therefore, in one embodiment, EXCS or a fragment or derivative thereof may be administered to a subject to treat or prevent a disorder associated with decreased expression or activity of EXCS. Examples of such disorders include, but are not limited to, an infection caused by a parasite classified as plasmodium or malaria-causing, parasitic entamoeba, leishmania, trypanosoma, toxoplasma, pneumocystis carinii, intestinal protozoa such as giardia, trichomonas, tissue nematode such as trichinella, intestinal nematode such as ascaris, lymphatic filarial nematode, trematode such as schistosoma, and cestode such as tapeworm an infection caused by a viral agent classified as adenovirus, arenavirus, bunyavirus, calicivirus, coronavirus, filovirus, hepadnavirus, herpesvirus, flavivirus, orthomyxovirus, parvovirus, papovavirus, paramyxovirus, picornavirus, poxvirus, reovirus, retrovirus, rhabdovirus, or togavirus; an infection caused by a bacterial agent classified as pneumococcus, staphylococcus, streptococcus, bacillus, corynebacterium, clostridium, meningococcus, gonococcus, listeria, moraxella, kingella, haemophilus, legionella, bordetella, gram-negative enterobacterium including shigella, salmonella, or campylobacter, pseudomonas, vibrio, brucella, francisella, yersinia, bartonella, norcardium, actinomyces, mycobacterium, spirochaetale, rickettsia, chlamydia, or mycoplasma; an infection caused by a fungal agent classified as aspergillus, blastomyces, dermatophytes, cryptococcus, coccidioides, malasezzia, histoplasma, or other mycosis-causing fungal agent; a gastrointestinal disorder such as dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease; prion diseases including kuru, Creutzfeldt-Jakob disease, and Gerstmann-Straussler-Scheinker syndrome; fatal familial insomnia, nutritional and metabolic diseases of the nervous system, neurofibromatosis, tuberous sclerosis, cerebeloretinal hemangioblastomatosis, encephalotrigeminal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis; inherited, metabolic, endocrine, and toxic myopathies; myasthenia gravis, periodic paralysis; mental disorders including mood, anxiety, and schizophrenic disorders; seasonal affective disorder (SAD); akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a reproductive disorder such as a disorder of prolactin production, infertility, including tubal disease, ovulatory defects, and endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, an ectopic pregnancy, and teratogenesis; cancer of the breast, fibrocystic breast disease, and galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, and gynecomastia; an autoimmune/inflammatory disorder such as inflammation, actinic keratosis, acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, bursitis, cirrhosis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, paroxysmal nocturnal hemoglobinemia, hepatitis, episodic lymphopenia with lymphocytotoxins, mixed connective tissue disease (MCTD), myelofibrosis, hypereosinophilia, irritable bowel syndrome, multiple sclerosis, myasthenia gravis, myocardial or pericardial inflammation, osteoarthritis, osteoporosis, pancreatitis, polymyositis, psoriasis, polycythemia vera, primary thrombocythemia, Reiter's syndrome, rheumatoid arthritis, scleroderma, Sjogren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma and hematopoietic cancer including lymphoma, leukemia, and myeloma, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus.

In another embodiment, a vector capable of expressing EXCS or a fragment or derivative thereof may be administered to a subject to treat or prevent a disorder associated with decreased expression or activity of EXCS including, but not limited to, those described above.

In a further embodiment, a pharmaceutical composition comprising a substantially purified EXCS in conjunction with a suitable pharmaceutical carrier may be adrinistered to a subject to treat or prevent a disorder associated with decreased expression or activity of EXCS including, but not limited to, those provided above.

In still another embodiment, an agonist which modulates the activity of EXCS may be administered to a subject to treat or prevent a disorder associated with decreased expression or activity of EXCS including, but not limited to, those listed above.

In a further embodiment, an antagonist of EXCS may be administered to a subject to treat or prevent a disorder associated with increased expression or activity of EXCS. Examples of such disorders include, but are not limited to, those infections and gastrointestinal, neurological, reproductive, autoimmune/inflammatory, and cell proliferative disorders including cancer described above. In one aspect, an antibody which specifically binds EXCS may be used directly as an antagonist or indirectly as a targeting or delivery mechanism for bringing a pharmaceutical agent to cells or tissues which express EXCS.

In an additional embodiment, a vector expressing the complement of the polynucleotide encoding EXCS may be administered to a subject to treat or prevent a disorder associated with increased expression or activity of EXCS including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of EXCS may be produced using methods which are generally known in the art. In particular, purified EXCS may be used to produce antibodies or to screen libraries of pharmaceutical agents to identify those which specifically bind EXCS. Antibodies to EXCS may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use.

For the production of antibodies, various hosts including goats, rabbits, rats, mice, humans, and others may be immunized by injection with EXCS or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to EXCS have an amino acid sequence consisting of at least about 5 amino acids, and generally will consist of at least about 10 amino acids. It is also preferable that these oligopeptides, peptides, or fragments are identical to a portion of the amino acid sequence of the natural protein and contain the entire amino acid sequence of a small, naturally occurring molecule. Short stretches of EXCS amino acids may be fused with those of another protein, such as KLH, and antibodies to the chimeric molecule may be produced.

Monoclonal antibodies to EXCS may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce EXCS-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for EXCS may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between EXCS and its specific antibody. A two-site, monoclonal-based immunoassay utilizing monoclonal antibodies reactive to two non-interfering EXCS epitopes is generally used, but a competitive binding assay may also be employed (Pound, supra).

Various methods such as Scatchard analysis in conjunction with radioimmunoassay techniques may be used to assess the affinity of antibodies for EXCS. Affinity is expressed as an association constant, Kₐ, which is defined as the molar concentration of EXCS-antibody complex divided by the molar concentrations of free antigen and free antibody under equilibrium conditions. The Kₐ determined for a preparation of polyclonal antibodies, which are heterogeneous in their affinities for multiple EXCS epitopes, represents the average affinity, or avidity, of the antibodies for EXCS. The Kₐ determined for a preparation of monoclonal antibodies, which are monospecific for a particular EXCS epitope, represents a true measure of affinity. High-affinity antibody preparations with Kₐ ranging from about 10⁹ to 10¹² L/mole are preferred for use in immunoassays in which the EXCS-antibody complex must withstand rigorous manipulations. Low-affinity antibody preparations with Kₐ ranging from about 10⁶ to 10⁷ L/mole are preferred for use in immunopurification and similar procedures which ultimately require dissociation of EXCS, preferably in active form, from the antibody (Catty, D. (1988) Antibodies, Volume I: A Practical Approach, IRL Press, Washington, D.C.; Liddell, J. E. and Cryer, A. (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of EXCS-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, the polynucleotides encoding EXCS, or any fragment or complement thereof, may be used for therapeutic purposes. In one aspect, the complement of the polynucleotide encoding EXCS may be used in situations in which it would be desirable to block the transcription of the mRNA. In particular, cells may be transformed with sequences complementary to polynucleotides encoding EXCS. Thus, complementary molecules or fragments may be used to modulate EXCS activity, or to achieve regulation of gene function. Such technology is now well known in the art, and sense or antisense oligonucleotides or larger fragments can be designed from various locations along the coding or control regions of sequences encoding EXCS.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors to express nucleic acid sequences complementary to the polynucleotides encoding EXCS. (See, e.g., Sambrook, supra; Ausubel, 1995, supra.)

Genes encoding EXCS can be turned off by transforming a cell or tissue with expression vectors which express high levels of a polynucleotide, or fragment thereof, encoding EXCS. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and may last even longer if appropriate replication elements are part of the vector system.

As mentioned above, modifications of gene expression can be obtained by designing complementary sequences or antisense molecules (DNA, RNA, or PNA) to the control, 5′, or regulatory regions of the gene encoding EXCS. Oligonucleotides derived from the transcription initiation site, e.g., between about positions −10 and +10 from the start site, may be employed. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding EXCS.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding EXCS. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a pharmaceutical or sterile composition, in conjunction with a pharmaceutically acceptable carrier, for any of the therapeutic effects discussed above. Such pharmaceutical compositions may consist of EXCS, antibodies to EXCS, and mimetics, agonists, antagonists, or inhibitors of EXCS. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs, or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing, Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, and succinic acids. Salts tend to be more soluble in aqueous or other protonic solvents than are the corresponding free base forms. In other cases, the preparation may be a lyophilized powder which may contain any or all of the following: 1 mM to 50 mM histidine, 0.1% to 2% sucrose, and 2% to 7% mannitol, at a pH range of 4.5 to 5.5, that is combined with buffer prior to use.

After pharmaceutical compositions have been prepared, they can be placed in an appropriate container and labeled for treatment of an indicated condition. For administration of EXCS, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models such as mice, rats, rabbits, dogs, or pigs. An animal model may also be used to determine the appropriate concentration range and route of administration. Such information can then be used to determine useful doses and routes for administration in humans.

A therapeutically effective dose refers to that amount of active ingredient, for example EXCS or fragments thereof, antibodies of EXCS, and agonists, antagonists or inhibitors of EXCS, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Pharmaceutical compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

### DIAGNOSTICS

In another embodiment, antibodies which specifically bind EXCS may be used for the diagnosis of disorders characterized by expression of EXCS, or in assays to monitor patients being treated with EXCS or agonists, antagonists, or inhibitors of EXCS. Antibodies useful for diagnostic purposes may be prepared in the same manner as described above for therapeutics. Diagnostic assays for EXCS include methods which utilize the antibody and a label to detect EXCS in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring EXCS, including ELISAs, RIAs, and FACS, are known in the art and provide a basis for diagnosing altered or abnormal levels of EXCS expression. Normal or standard values for EXCS expression are established by combining body fluids or cell extracts taken from normal mammalian subjects, for example, human subjects, with antibody to EXCS under conditions suitable for complex formation. The amount of standard complex formation may be quantitated by various methods, such as photometric means. Quantities of EXCS expressed in subject, control, and disease samples from biopsied tissues are compared with the standard values. Deviation between standard and subject values establishes the parameters for diagnosing disease.

In another embodiment of the invention, the polynucleotides encoding EXCS may be used for diagnostic purposes. The polynucleotides which may be used include oligonucleotide sequences, complementary RNA and DNA molecules, and PNAs. The polynucleotides may be used to detect and quantify gene expression in biopsied tissues in which expression of EXCS may be correlated with disease. The diagnostic assay may be used to determine absence, presence, and excess expression of EXCS, and to monitor regulation of EXCS levels during therapeutic intervention.

In one aspect, hybridization with PCR probes which are capable of detecting polynucleotide sequences, including genomic sequences, encoding EXCS or closely related molecules may be used to identify nucleic acid sequences which encode EXCS. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding EXCS, allelic variants, or related sequences.

Probes may also be used for the detection of related sequences, and may have at least 50% sequence identity to any of the EXCS encoding sequences. The hybridization probes of the subject invention may be DNA or RNA and may be derived from the sequence of SEQ ID NO:27-52 or from genomic sequences including promoters, enhancers, and introns of the EXCS gene.

Means for producing specific hybridization probes for DNAs encoding EXCS include the cloning of polynucleotide sequences encoding EXCS or EXCS derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding EXCS may be used for the diagnosis of disorders associated with expression of EXCS. Examples of such disorders include, but are not limited to, an infection caused by a parasite classified as plasmodium or malaria-causing, parasitic entamoeba, leishmania, trypanosoma, toxoplasma, pneumocystis carinii, intestinal protozoa such as giardia, trichomonas, tissue nematode such as trichinella, intestinal nematode such as ascaris, lymphatic filarial nematode, trematode such as schistosoma, and cestode such as tapeworm an infection caused by a viral agent classified as adenovirus, arenavirus, bunyavirus, calicivirus, coronavirus, filovirus, hepadnavirus, herpesvirus, flavivirus, orthomyxovirus, parvovirus, papovavirus, paramyxovirus, picornavirus, poxvirus, reovirus, retrovirus, rhabdovirus, or togavirus; an infection caused by a bacterial agent classified as pneumococcus, staphylococcus, streptococcus, bacillus, corynebacterium, clostridium, meningococcus, gonococcus, listeria, moraxella, kingella, haemophilus, legionella, bordetella, gram-negative enterobacterium including shigella, salmonella, or campylobacter, pseudomonas, vibrio, brucella, francisella, yersinia, bartonella, norcardium, actinomyces, mycobacterium, spirochaetale, rickettsia, chlamydia, or mycoplasma; an infection caused by a fungal agent classified as aspergillus, blastomyces, dermatophytes, cryptococcus, coccidioides, malasezzia, histoplasma, or other mycosis-causing fungal agent; a gastrointestinal disorder such as dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha -antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease; prion diseases including kuru, Creutzfeldt-Jakob disease, and Gerstmann-Straussler-Scheinker syndrome; fatal familial insomnia, nutritional and metabolic diseases of the nervous system, neurofibromatosis, tuberous sclerosis, cerebeloretinal hemangioblastomatosis, encephalotrigeminal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis; inherited, metabolic, endocrine, and toxic myopathies; myasthenia gravis, periodic paralysis; mental disorders including mood, anxiety, and schizophrenic disorders; seasonal affective disorder (SAD); akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a reproductive disorder such as a disorder of prolactin production, infertility, including tubal disease, ovulatory defects, and endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, an ectopic pregnancy, and teratogenesis; cancer of the breast, fibrocystic breast disease, and galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, and gynecomastia; an autoimmune/inflammatory disorder such as inflammation, actinic keratosis, acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, bursitis, cirrhosis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, paroxysmal nocturnal hemoglobinemia, hepatitis, episodic lymphopenia with lymphocytotoxins, mixed connective tissue disease (MCTD), myelofibrosis, hypereosinophilia, irritable bowel syndrome, multiple sclerosis, myasthenia gravis, myocardial or pericardial inflammation, osteoarthritis, osteoporosis, pancreatitis, polymyositis, psoriasis, polycythemia vera, primary thrombocythemia, Reiter's syndrome, rheumatoid arthritis, scleroderma, Sjogren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma and hematopoietic cancer including lymphoma, leukemia, and myeloma, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. The polynucleotide sequences encoding EXCS may be used in Southern or northern analysis, dot blot, or other membrane-based technologies; in PCR technologies; in dipstick, pin, and multiformat ELISA-like assays; and in microarrays utilizing fluids or tissues from patients to detect altered EXCS expression. Such qualitative or quantitative methods are well known in the art.

In a particular aspect, the nucleotide sequences encoding EXCS may be useful in assays that detect the presence of associated disorders, particularly those mentioned above. The nucleotide sequences encoding EXCS may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding EXCS in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of EXCS, a normal or standard profile for expression is established. This may be accomplished by combining body fluids or cell extracts taken from normal subjects, either animal or human, with a sequence, or a fragment thereof, encoding EXCS, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding EXCS may involve the use of PCR. These oligomers may be chemically synthesized, generated enzymatically, or produced in vitro. Oligomers will preferably contain a fragment of a polynucleotide encoding EXCS, or a fragment of a polynucleotide complementary to the polynucleotide encoding EXCS, and will be employed under optimized conditions for identification of a specific gene or condition. Oligomers may also be employed under less stringent conditions for detection or quantification of closely related DNA or RNA sequences.

Methods which may also be used to quantify the expression of EXCS include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples may be accelerated by running the assay in a high-throughput format where the oligomer of interest is presented in various dilutions and a spectrophotometric or calorimetric response gives rapid quantitation.

In further embodiments, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.)

In another embodiment of the invention, nucleic acid sequences encoding EXCS may be used to generate hybridization probes useful in mapping the naturally occurring genomic sequence. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.)

Fluorescent in situ hybridization (FISH) may be correlated with other physical chromosome mapping techniques and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding EXCS on a physical chromosomal map and a specific disorder, or a predisposition to a specific disorder, may help define the region of DNA associated with that disorder. The nucleotide sequences of the invention may be used to detect differences in gene sequences among normal, carrier, and affected individuals.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the number or arm of a particular human chromosome is not known. New sequences can be assigned to chromosomal arms by physical mapping. This provides valuable information to investigators searching for disease genes using positional cloning or other gene discovery techniques. Once the disease or syndrome has been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the subject invention may also be used to detect differences in the chromosomal location due to translocation, inversion, etc., among normal, carrier, or affected individuals.

In another embodiment of the invention, EXCS, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between EXCS and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with EXCS, or fragments thereof, and washed. Bound EXCS is then detected by methods well known in the art. Purified EXCS can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding EXCS specifically compete with a test compound for binding EXCS. In this manner, antibodies can be used to detect the presence of any peptide which shares one or more antigenic determinants with EXCS.

In additional embodiments, the nucleotide sequences which encode EXCS may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The disclosures of all patents, applications and publications, mentioned above and below, in particular U.S. Ser. No. 60/134,949, U.S. Ser. No. 60/144,270, U.S. Ser. No. 60/146,700, and U.S. Ser. No. 60/157,508, are hereby expressly incorporated by reference.

### EXAMPLES

I. Construction of cDNA Libraries

RNA was purchased from Clontech or isolated from tissues described in Table 4. Some tissues were homogenized and lysed in guanidinium isothiocyanate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A+) RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art. (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasmid (Stratagene), PSPORT 1 plasmid (Life Technologies), pcDNA2.1 plasmid (Invitrogen, Carlsbad Calif.), or pINCY plasmid (Incyte Pharmaceuticals, Palo Alto Calif.). Recombinant plasmids were transformed into competent E. coli cells including XL1-Blue, XL1-BlueMRF, or SOLR from Stratagene or DH5α, DH10B, or ElectroMAX DH10B from Life Technologies.

II. Isolation of cDNA Clones

Plasmids were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Ore.) and a FLUOROSKAN II fluorescence scanner (Labsystems Oy, Helsinki, Finland).

III. Sequencing and Analysis

cDNA sequencing reactions were processed using standard methods or high-throughput instrumentation such as the ABI CATALYST 800 (Perkin-Elmer) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Pharmacia Biotech or supplied in ABI sequencing kits such as the ABI PRISM BIGDYE Terminator cycle sequencing ready reaction kit (Perkin-Elmer). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Perkin-Elmer) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example V.

The polynucleotide sequences derived from cDNA sequencing were assembled and analyzed using a combination of software programs which utilize algorithms well known to those skilled in the art. Table 5 summarizes the tools, programs, and algorithms used and provides applicable descriptions, references, and threshold parameters. The first column of Table 5 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score, the greater the homology between two sequences). Sequences were analyzed using MACDNASIS PRO software (Hitachi Software Engineering, South San Francisco Calif.) and LASERGENE software (DNASTAR). Polynucleotide and polypeptide sequence alignments were generated using the default parameters specified by the clustal algorithm as incorporated into the MEGALIGN multisequence alignment program (DNASTAR), which also calculates the percent identity between aligned sequences.

The polynucleotide sequences were validated by removing vector, linker, and polyA sequences and by masking ambiguous bases, using algorithms and programs based on BLAST, dynamic programing, and dinucleotide nearest neighbor analysis. The sequences were then queried against a selection of public databases such as the GenBank primate, rodent, mammalian, vertebrate, and eukaryote databases, and BLOCKS, PRINTS, DOMO, PRODOM, and PFAM to acquire annotation using programs based on BLAST, FASTA, and BLIMPS. The sequences were assembled into full length polynucleotide sequences using programs based on Phred, Phrap, and Consed, and were screened for open reading frames using programs based on GeneMark, BLAST, and FASTA. The full length polynucleotide sequences were translated to derive the corresponding full length amino acid sequences, and these full length sequences were subsequently analyzed by querying against databases such as the GenBank databases (described above), SwissProt, BLOCKS, PRINTS, DOMO, PRODOM, Prosite, and Hidden Markov Model (HMM)-based protein family databases such as PFAM. HMM is a probabilistic approach which analyzes consensus primary structures of gene families. (See, e.g., Eddy, S. R. (1996) Curr. Opin. Struct. Biol. 6:361-365.)

The programs described above for the assembly and analysis of full length polynucleotide and amino acid sequences were also used to identify polynucleotide sequence fragments from SEQ ID NO:27-52. Fragments from about 20 to about 4000 nucleotides which are useful in hybridization and amplification technologies were described in The Invention section above.

IV. Northern Analysis

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel, 1995, supra, ch. 4 and 16.)

Analogous computer techniques applying BLAST were used to search for identical or related molecules in nucleotide databases such as GenBank or LIFESEQ (Incyte Pharmaceuticals). This analysis is much faster than multiple membrane-based hybridizations. In addition, the sensitivity of the computer search can be modified to determine whether any particular match is categorized as exact or similar. The basis of the search is the product score, which is defined as:

$$\frac{\% sequence identity \times \% maximum BLAST score}{100}$$

The product score takes into account both the degree of similarity between two sequences and the length of the sequence match. For example, with a product score of 40, the match will be exact within a 1% to 2% error, and, with a product score of 70, the match will be exact. Similar molecules are usually identified by selecting those which show product scores between 15 and 40, although lower scores may identify related molecules.

The results of northern analyses are reported as a percentage distribution of libraries in which the transcript encoding EXCS occurred. Analysis involved the categorization of cDNA libraries by organ/tissue and disease. The organ/tissue categories included cardiovascular, dermatologic, developmental, endocrine, gastrointestinal, hematopoietic/immune, musculoskeletal, nervous, reproductive, and urologic. The disease/condition categories included cancer, inflammation, trauma, cell proliferation, neurological, and pooled. For each category, the number of libraries expressing the sequence of interest was counted and divided by the total number of libraries across all categories. Percentage values of tissue-specific and disease- or condition-specific expression are reported in Table 3.

V. Chromosomal Mapping of EXCS Encoding Polynucleotides

The cDNA sequences which were used to assemble SEQ ID NO:45-52 were compared with sequences from the Incyte LIFESEQ database and public domain databases using BLAST and other implementations of the Smith-Waterman algorithm. Sequences from these databases that matched SEQ ID NO:27-52 were assembled into clusters of contiguous and overlapping sequences using assembly algorithms such as Phrap (Table 5). Radiation hybrid and genetic mapping data available from public resources such as the Stanford Human Genome Center (SHGC), Whitehead Institute for Genome Research (WIGR), and Genethon were used to determine if any of the clustered sequences had been previously mapped. Inclusion of a mapped sequence in a cluster resulted in the assignment of all sequences of that cluster, including its particular SEQ ID NO:, to that map location.

The genetic map location of SEQ ID NO:47 is described in The Invention as a range, or interval, of a human chromosome. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Genethon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VI. Extension of EXCS Encoding Polynucleotides

The full length nucleic acid sequences of SEQ ID NO:27-52 were produced by extension of an appropriate fragment of the full length molecule using oligonucleotide primers designed from this fragment. One primer was synthesized to initiate 5′ extension of the known fragment, and the other primer, to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and β-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Ore.) dissolved in 1× TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Pharmacia Biotech), treated with Pfu DNA polymerase (Stratagene) to fill-in restriction site overhangs, and transfected into competent E. coli cells. Transformed cells were selected on antibiotic-containing media, individual colonies were picked and cultured overnight at 37° C. in 384-well plates in LB/2× carb liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Perkin-Elmer).

In like manner, the nucleotide sequences of SEQ ID NO:27-52 are used to obtain 5′ regulatory sequences using the procedure above, oligonucleotides designed for such extension, and an appropriate genomic library.

VII. Labeling and Use of Individual Hybridization Probes

Hybridization probes derived from SEQ ID NO:27-52 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Pharmacia Biotech), and T4 polynucleotide kinase (DuPont NEN, Boston Mass.). The labeled oligonucleotides are substantially purified using a SEPHADEX G-25 superfine size exclusion dextran bead column (Amersham Pharmacia Biotech). An aliquot containing 107 counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

VIII. Microarrays

A chemical coupling procedure and an ink jet device can be used to synthesize array elements on the surface of a substrate. (See, e.g., Baldeschweiler, supra.) An array analogous to a dot or slot blot may also be used to arrange and link elements to the surface of a substrate using thermal, UV, chemical, or mechanical bonding procedures. A typical array may be produced by hand or using available methods and machines and contain any appropriate number of elements. After hybridization, nonhybridized probes are removed and a scanner used to determine the levels and patterns of fluorescence. The degree of complementarity and the relative abundance of each probe which hybridizes to an element on the microarray may be assessed through analysis of the scanned images.

Full-length cDNAs, Expressed Sequence Tags (ESTs), or fragments thereof may comprise the elements of the microarray. Fragments suitable for hybridization can be selected using software well known in the art such as LASERGENE software (DNASTAR). Full-length cDNAs, ESTs, or fragments thereof corresponding to one of the nucleotide sequences of the present invention, or selected at random from a cDNA library relevant to the present invention, are arranged on an appropriate substrate, e.g., a glass slide. The cDNA is fixed to the slide using, e.g., UV cross-linking followed by thermal and chemical treatments and subsequent drying. (See, e.g., Schena, M. et al. (1995) Science 270:467470; Shalon, D. et al. (1996) Genome Res. 6:639-645.) Fluorescent probes are prepared and used for hybridization to the elements on the substrate. The substrate is analyzed by procedures described above.

IX. Complementary Polynucleotides

Sequences complementary to the EXCS-encoding sequences, or any parts thereof, are used to detect, decrease, or inhibit expression of naturally occurring EXCS. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of EXCS. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the EXCS-encoding transcript.

X. Expression of EXCS

Expression and purification of EXCS is achieved using bacterial or virus-based expression systems. For expression of EXCS in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express EXCS upon induction with isopropyl beta-D-thiogalactopyranoside (IPTG). Expression of EXCS in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding EXCS by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, EXCS is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Pharmacia Biotech). Following purification, the GST moiety can be proteolytically cleaved from EXCS at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified EXCS obtained by these methods can be used directly in the following activity assay.

XI. Demonstration of EXCS Activity

EXCS activity is measured by one of several methods. Growth factor activity is measured by the stimulation of DNA synthesis in Swiss mouse 3T3 cells. (McKay, I. and Leigh, I., eds. (1993) Growth Factors: A Practical Approach, Oxford University Press, New York, N.Y.) Initiation of DNA synthesis indicates the cells' entry into the mitotic cycle and their commitment to undergo later division. 3T3 cells are competent to respond to most growth factors, not only those that are mitogenic, but also those that are involved in embryonic induction. This competence is possible because the in vivo specificity demonstrated by some growth factors is not necessarily inherent but is determined by the responding tissue. In this assay, varying amounts of EXCS are added to quiescent 3T3 cultured cells in the presence of [³H]thymidine, a radioactive DNA precursor. EXCS for this assay can be obtained by recombinant means or from biochemical preparations. Incorporation of [³H]thymidine into acid-precipitable DNA is measured over an appropriate time interval, and the amount incorporated is directly proportional to the amount of newly synthesized DNA. A linear dose-response curve over at least a hundred-fold EXCS concentration range is indicative of growth factor activity. One unit of activity per milliliter is defined as the concentration of EXCS producing a 50% response level, where 100% represents maximal incorporation of [³H]thymidine into acid-precipitable DNA.

Alternatively, an assay for cytokine activity measures the proliferation of cultured cells such as fibroblasts or leukocytes. In this assay, the amount of tritiated thymidine incorporated into newly synthesized DNA is used to estimate proliferative activity. Varying amounts of EXCS are added to cultured fibroblasts, or cultured leukocytes such as granulocytes, monocytes, or lymphocytes, in the presence of [³H]thymidine, a radioactive DNA precursor. EXCS for this assay can be obtained by recombinant means or from biochemical preparations. Incorporation of [³H]thymidine into acid-precipitable DNA is measured over an appropriate time interval, and the amount incorporated is directly proportional to the amount of newly synthesized DNA. A linear dose-response curve over at least a hundred-fold EXCS concentration range is indicative of EXCS activity. One unit of activity per milliliter is conventionally defined as the concentration of EXCS producing a 50% response level, where 100% represents maximal incorporation of [³H]thymidine into acid-precipitable DNA.

An alternative assay for EXCS cytokine activity utilizes a Boyden micro chamber (Neuroprobe, Cabin John, Md.) to measure leukocyte chemotaxis. In this assay, about 10⁵ migratory cells such as macrophages or monocytes are placed in cell culture media in the upper compartment of the chamber. Varying dilutions of EXCS are placed in the lower compartment. The two compartments are separated by a 5 or 8 micron pore polycarbonate filter (Nucleopore, Pleasanton Calif.). After incubation at 37° C. for 80 to 120 minutes, the filters are fixed in methanol and stained with appropriate labeling agents. Cells which migrate to the other side of the filter are counted using standard microscopy. The chemotactic index is calculated by dividing the number of migratory cells counted when EXCS is present in the lower compartment by the number of migratory cells counted when only media is present in the lower compartment. The chemotactic index is proportional to the activity of EXCS.

Alternatively, cell lines or tissues transformed with a vector containing nucleotide sequences encoding EXCS can be assayed for EXCS activity by immunoblotting. Cells are denatured in SDS in the presence of β-mercaptoethanol, nucleic acids removed by ethanol precipitation, and proteins purified by acetone precipitation. Pellets are resuspended in 20 mM tris buffer at pH 7.5 and incubated with Protein G-Sepharose pre-coated with an antibody specific for EXCS. After washing, the Sepharose beads are boiled in electrophoresis sample buffer, and the eluted proteins subjected to SDS-PAGE. The SDS-PAGE is transferred to a nitrocellulose membrane for immunoblotting, and the EXCS activity is assessed by visualizing and quantifying bands on the blot using the antibody specific for EXCS as the primary antibody and ¹²⁵I-labeled IgG specific for the primary antibody as the secondary antibody.

XII. Functional Assays

EXCS function is assessed by expressing the sequences encoding EXCS at physiologically elevated levels in mammalian cell culture systems. cDNA is subcloned into a mammalian expression vector containing a strong promoter that drives high levels of cDNA expression. Vectors of choice include pCMV SPORT plasmid (Life Technologies) and pCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of EXCS on gene expression can be assessed using highly purified populations of cells transfected with sequences encoding EXCS and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding EXCS and other genes of interest can be analyzed by northern analysis or microarray techniques.

XII. Production of EXCS Specific Antibodies

EXCS substantially purified using polyacrylamide gel electrophoresis (PAGE; see, e.g., Harrington, M. G. (1990) Methods Enzymol. 182:488-495), or other purification techniques, is used to immunize rabbits and to produce antibodies using standard protocols.

Alternatively, the EXCS amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, ch. 11.)

Typically, oligopeptides of about 15 residues in length are synthesized using an ABI 431A peptide synthesizer (Perkin-Elmer) using fmoc-chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide and anti-EXCS activity by, for example, binding the peptide or EXCS to a substrate, blocking with 1% BSA, reacting with rabbit antisera, washing, and reacting with radio-iodinated goat anti-rabbit IgG.

XIV. Purification of Naturally Occurring EXCS Using Specific Antibodies

Naturally occurring or recombinant EXCS is substantially purified by immunoaffinity chromatography using antibodies specific for EXCS. An immunoaffinity column is constructed by covalently coupling anti-EXCS antibody to an activated chromatographic resin, such as CNBr-activated SEPHAROSE (Amersham Pharmacia Biotech). After the coupling, the resin is blocked and washed according to the manufacturer's instructions.

Media containing EXCS are passed over the immunoaffinity column, and the column is washed under conditions that allow the preferential absorbance of EXCS (e.g., high ionic strength buffers in the presence of detergent). The column is eluted under conditions that disrupt antibody/EXCS binding (e.g., a buffer of pH 2 to pH 3, or a high concentration of a chaotrope, such as urea or thiocyanate ion), and EXCS is collected.

XV. Identification of Molecules Which Interact with EXCS

EXCS, or biologically active fragments thereof, are labeled with ¹²⁵I Bolton-Hunter reagent. (See, e.g., Bolton A. E. and W. M. Hunter (1973) Biochem. J. 133:529-539.) Candidate molecules previously arrayed in the wells of a multi-well plate are incubated with the labeled EXCS, washed, and any wells with labeled EXCS complex are assayed. Data obtained using different concentrations of EXCS are used to calculate values for the number, affinity, and association of EXCS with the candidate molecules.

Alternatively, molecules interacting with EXCS are analyzed using the yeast two-hybrid system as described in Fields, S. and O. Song (1989, Nature 340:245-246), or using commercially available kits based on the two-hybrid system, such as the MATCHMAKER system (Clontech).

Various modifications and variations of the described methods and systems of the invention will be apparent to those skilled in the art without departing from the scope and spirit of the invention. Although the invention has been described in connection with certain embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1

| Polypeptide | Nucleotide | | | |
|---|---|---|---|---|
| SEQ ID NO: | SEQ ID NO: | Clone ID | Library | Fragments |
| 1 | 27 | 1288847 | BRAINOT11 | 00334U1(U937NOT01), 840916T6 (PROSTUT05), |
| | | | | 1288847F6 (BRAINOT11), 1288847H1 (BRAINOT11), |
| | | | | 1651772F6 (PROSTUT08), 2720131F6 (LUNGTUT10), |
| | | | | 2954659F6 (KIDNFET01), 3321171H1 (PTHYNOT03), |
| | | | | 3520878T6 (LUNGNON03), 3870826H1 (BMARNOT03), |
| | | | | 5271406H1 (OVARDIN02), SBYA00334U1 |
| 2 | 28 | 1329044 | PANCNOT07 | 04082U1 (HMC1NOT01), 1329044F1 (PANCNOT07), |
| | | | | 1329044H1 (PANCNOT07), 1329044T1 (PANCNOT07), |
| | | | | SBYA04082U1 |
| 3 | 29 | 1493630 | PROSNON01 | 1493630H1 (PROSNON01), 1493630R6 (PROSNON01), |
| | | | | 1493630T1 (PROSNON01) |
| 4 | 30 | 1533041 | SPLNNOT04 | 1533041F1 (SPLNNOT04), 1533041H1 (SPLNNOT04), |
| | | | | 2688779F6 (LUNGNOT23), 3973608H1 (ADRETUT06), |
| 5 | 31 | 1566162 | HEALDIT02 | 1566162H1 (HEALDIT02), 1759922T6 (PITUNOT03), |
| 6 | 32 | 1811831 | PROSTUT12 | 1811831F6 (PROSTUT12), 1811831H1 (PROSTUT12) |
| 7 | 33 | 1835447 | BRAINON01 | 1835447H1 (BRAINON01), 1835447R6 (BRAINON01), |
| | | | | 4523747H1 (HNT2TXT01), 5310808H1 (KIDETXS02) |
| 8 | 34 | 3892281 | BRSTTTUT16 | 1948957R6 (PITUNOT01), 3892281H1 (BRSTTUT16), |
| | | | | 3895852T6 (TLYMNOT05) |
| 9 | 35 | 4318494 | BRADDIT02 | 4318494F6 (BRADDIT02), 4318494H1 (BRADDIT02), |
| | | | | 4318494T6 (BRADDIT02) |
| 10 | 36 | 5090841 | UTRSTMR01 | 742729H1 (PANCNOT04), 1329245H1 (PANCNOT07), |
| | | | | 4539309H1 (THYRTMT01), 590841F6 (UTRSTMR01), |
| | | | | 5090841H1 (UTRSTMR01), 5153892H1 (HEARFET03) |
| 11 | 37 | 2006548 | TESTNOT03 | 1725329X11C1 (PROSNOT14), 2006548H1 (TESTNOT03), |
| | | | | 3476792F6 (OVARNOT11), SBIA08125D1, SBIA01870D1 |
| 12 | 38 | 2207183 | SINTFET03 | 191932F1 (SYNORAB01), 1273270F1 (TESTTUT02), |
| | | | | 2207183H1 (SINTFET03), 2219907H1 (LUNGNOT18), |
| | | | | 3336344H1 (SPLNNOT10) |
| 13 | 39 | 2267403 | UTRSNOT02 | 1449035F1 and 1449035R1 (PLACNOT02), 1599756F6 |
| | | | | (BLADNOT03), 2267403H1 and 2267403R6 (UTRSNOT02), |
| | | | | 3145756F6 (TESTNOT07) |
| 14 | 40 | 2933038 | THYMNON04 | 157761F1 (THP1PLB02), 2933038H1 (THYMNON04), |
| | | | | 3294396F6 (TLYJINT01) |
| 15 | 41 | 3216587 | TESTNOT07 | 3216587F and 3216587H2 (TESTNOT07), 3416261H1 |
| | | | | (PTHYNOT04), 4204275F6 (BRAITUT29), 4316562F6 |
| | | | | (BRAFNOT01), 5385916H1 (BRAINOT19) |
| 16 | 42 | 5037143 | LIVRTUT13 | 5035406H1, 5037143H1, and 5037690T6 (LIVRTUT13) |
| 17 | 43 | 1235265 | LUNGFET03 | 523352F1, (MMLR2DT01), 1262491R1 (SYNORAT05), |
| | | | | 1699607F6 (BLADTUT05), 1717617H1, (UCMCNOT02), |
| | | | | 1720254F6 (BLADNOT06), SBLA02298F1 |
| 18 | 44 | 5571181 | TLYMNOT08 | 4348184T6 (TLYMTXT01), 4905349F6 (TLYMNOT08), |
| | | | | 5571181H1 (TLYMNOT08) |
| 19 | 45 | 685374 | UTRSNOT02 | 111201R6 (PITUNOT01), 685374H1 (UTRSNOT02), 685374R6 |
| | | | | (UTRSNOT02), 837768R1 (PROSNOT07), 1369176R6 |
| | | | | (SCORNON02), 3321269H1 (PTHYNOT03), 4309489H1 |
| | | | | (BRAUNOT01), 4943366F6 (BRAIFEN05), 5108512H1 |
| | | | | (PROSTUS19) |
| 20 | 46 | 843193 | PROSTUT05 | 843193H1 (PROSTUT05), 843193X23 (PROSTIT05), |
| | | | | 843193X25 (PROSTUT05), 996669R6 (KIDNTUT01), |
| | | | | 4699738F6 (BRALNOT01), SZAL00006D1 |
| 21 | 47 | 1359783 | LUNGNOT12 | 1359783F1 (LUNGNOT12), 1403716H1 (LATRTUT02), |
| | | | | 2160063H1 (ENDCNOT02), 2464542H1 (THYRNOT08), |
| | | | | 3423249H1 (UCMCNOT04), SANA00380F1, SANA02266F1, |
| | | | | SANA02708F1, SANA00364F1, SANA01088F1, SANA03698F1 |
| 22 | 48 | 1440015 | THYRNOT03 | 1440015H1 (THYRNOT03), 1462822H1 (PANCNOT04), |
| | | | | 1577577F6 (LNODNOT03) |
| 23 | 49 | 1652885 | PROSTUT08 | 1478195T1 (CORPNOT02), 1652885F6 (PROSTUT08), |
| | | | | 1652885H1 (PROSTUT08) |
| 24 | 50 | 4003984 | HNT2AZS07 | 4003984H1 (HNT2AZS07), 4003984R6 (HNT2AZS07), |
| | | | | 4003984T6 (HNT2AZS07) |
| 25 | 51 | 4365383 | SKIRNOT01 | 4365383F6 (SKIRNOT01), 4365383H1 (SKIRNOT01), |
| | | | | 5098601H2 (EPIMNON05), g3228929 |
| 26 | 52 | 5497814 | BRABDIR01 | 5497814F6 (BRABDIR01), 5497814H1 (BRABDIR01) |

TABLE 2

| | | Potential | Potential | | | Analytical |
|---|---|---|---|---|---|---|
| Polypeptide | Amino Acid | Phosphorylation | Glycosylation | Signature | Homologous | Methods and |
| SEQ ID NO: | Residues | Sites | Sites | Sequence | Sequences | Databases |
| 1 | 77 | S62 | | Signal peptide: | | SPSCAN |
| | | | | M1-T20 | | HMM |
| 2 | 88 | T3 S23 T59 S65 | | Signal peptide: | | SPSCAN |
| | | | | M1-E26 or M1-S27 | | HMM |
| 3 | 96 | S20 S83 S91 | | Signal peptide: | | SPSCAN |
| | | | | M1-G21 or M1-C22 | | HMM |
| 4 | 104 | S45 S90 | | Signal peptide: | | SPSCAN |
| | | | | M1-S30 or M1-C26 | | HMM |
| 5 | 60 | S19 | | Signal peptide: | | SPSCAN |
| | | | | M1-S19 | | HMM |
| 6 | 117 | T105 S40 S112 | | Signal peptide: | | SPSCAN |
| | | S40 | | M1-A28 or M1-A31 | | HMM |
| 7 | 86 | S26 | | Signal peptide: | | SPSCAN |
| | | | | M1-A24 or M1-P22 | | HMM |
| | | | | ATP/GTP binding | | MOTIFS |
| | | | | site: G45-T52 | | |
| 8 | 109 | S27 S69 S51 | | Signal peptide: | | SPSCAN |
| | | | | M1-G23 or M1-A29 | | HMM |
| 9 | 111 | S45 S52 S74 | | Signal peptide: | | SPSCAN |
| | | | | M1-S20 | | HMM |
| 10 | 182 | T161 S125 T148 | | Signal peptide: | | SPSCAN |
| | | | | M1-A34 or M1-S31 | | HMM |
| 11 | 105 | T15 S64 Y94 | | Signal peptide: | Venom protein A: | BLAST-GenBank |
| | | | | M1-I22 | (P25687) | BLAST-SwissProt |
| | | | | Venom Protein A | g6524951 Bv8 | MOTIFS |
| | | | | A20-C96 | variant 3 | SPScan |
| | | | | | precursor | BLAST-PRODOM |
| | | | | | | HMMER |
| 12 | 342 | S48 S50 T61 | N157 N192 N270 | Transmembrane | g4689122 HSPC013 | BLAST-GenBank |
| | | T167 S194 S255 | N281 | Domain: L314-T334 | hematopoietic | MOTIFS |
| | | S14 S39 S74 | | EGF-like domain | stem/progenitor | HMMER |
| | | T225 T334 | | cysteine pattern | cells | BLAST - DOMO |
| | | | | signature: C294- | | |
| | | | | C305 | | |
| | | | | EGF domain: D258- | | |
| | | | | Q308 | | |
| 13 | 451 | S41 T62 S125 | N114 | Signal Peptide: | g4808227 | BLAST-GenBank |
| | | S142 T154 S182 | | M1-A25 | C-terminal part | MOTIFS |
| | | S260 T281 S398 | | ATP/GTP binding | of a Chordin-like | SPScan |
| | | S444 T239 S328 | | site motif A (P- | protein | HMMER-PFAM, |
| | | T416 | | loop): A251 - T258 | | HMMER |
| | | | | von Willebrand | | BLIMPS - PFAM |
| | | | | factor type C | | |
| | | | | domains: C33-C95, | | |
| | | | | C111-C174, C252- | | |
| | | | | C314 | | |
| 14 | 189 | T64 S37 | | Signal Peptide: | Y29783 Human | MOTIFS |
| | | | | M1-P23 | interleukin B30 | SPScan |
| | | | | Interleukin-6/G- | | HMMER |
| | | | | CSF: T65-F109 | | BLIMPS-BLOCKS |
| | | | | S151-A181 | | BLIMPS-PRINTS |
| | | | | | | BLAST-GENESEQ |
| 15 | 216 | S19 T49 T122 | N47 | Recoverin Family | Calsenilin | BLAST-GenBank |
| | | T191 S198 T49 | | Signature: H34- | (g4416432) | MOTIFS |
| | | T73 S105 T170 | | F48, F48-G67, | A-Type potassium | HMMER-PFAM |
| | | | | L94-L115, L118- | channel | BLIMPS-PRINTS |
| | | | | N137, G140-M158 | modulatory | |
| | | | | P164-F179, V190- | protein 1 | |
| | | | | L210 | (g6969255) | |
| | | | | EF-hand Domains: | | |
| | | | | K126-I154, H174- | | |
| | | | | D202 | | |
| 16 | 178 | T111 S174 T124 | | Signal Peptide: | Pancreatic | BLAST-GenBank |
| | | | | M1-G29 | polypeptide | MOTIFS |
| | | | | Pancreatic | precursor | HMMER |
| | | | | hormone peptide: | (g190270) | SPScan |
| | | | | A30-C65 | | ProfileScan |
| | | | | Pancreatic | | HMMER-PFAM |
| | | | | hormone | | BLAST-PRODOM |
| | | | | precursor: G149- | | BLIMPS-PRINTS |
| | | | | L178 | | BLIMPS-BLOCKS |
| 17 | 177 | S168 T22 S43 | N5 | Signal peptide: | Fibrosin | MOTIFS |
| | | S73 S115 S175 | | M1-A60 | (g170336) MUS | BLAST_GenBank |
| | | | | | musculus | SPSCAN |
| 18 | 179 | S64 S84 T99 T53 | N54 N68 N97 | Signal peptide: | g6996554 TIF | BLAST_GENBANK |
| | | S86 S108 S126 | | M1-A33 | alpha-protein | MOTIFS |
| | | T151 S173 | | Transmembrane | | SPSCAN |
| | | | | domain: V8-L27 | | HMMR |
| 19 | 213 | S7 T39 S93 S155 | N189 N202 | G178-S185: | g4323515 | Motifs |
| | | S187 S112 Y46 | | ATP/GTP binding | Fibroblast growth | BLAST_GENBANK |
| | | | | site | factor 13 isoform | HMMER_PFAM |
| | | | | Fibroblast growth | 1B | PROFILESCAN |
| | | | | factors: K14-P145 | | BLIMPS_BLOCKS |
| | | | | HBGF/FGF family | | BLIMPS_PRINTS |
| | | | | signature: V58- | | BLAST_PRODOM |
| | | | | S112, W116-P143 | | BLAST_DOMO |
| 20 | 239 | S97 T99 T45 S71 | N160 | HBGF/FGF family | G3041790 | Motifs |
| | | S85 T92 S127 | | signature: Q74- | Fibroblast growth | BLAST_GENBANK |
| | | S144 T226 T232 | | L201 | factor FGF-17 | SPSCAN |
| | | Y81 Y130 | | Signal peptide: | | HMMER_PFAM |
| | | | | M1-P15 | | PROFILESCAN |
| | | | | IL1/HBGF Family | | BLIMPS_BLOCKS |
| | | | | Signature: D149 | | BLAST_PRINTS |
| | | | | H169 | | BLAST_PRODOM |
| | | | | | | BLAST_DOMO |
| 21 | 493 | T251 T331 T340 | N249 | Signal peptide: | g2429083 | Motifs |
| | | S430 T132 S154 | | M1-S17 | T16 EGF-like | BLAST-GENBANK |
| | | S287 S399 S402 | | EGF-like domain: | protein | SPSCAN |
| | | T425 T475 | | C224-R299 | | HMMER |
| | | | | | | BLIMPS_BLOCKS |
| | | | | | | BLAST_DOMO |
| 22 | 121 | S76 S76 | | Signal peptide: | g 189228 | Motifs |
| | | | | M1-D30 Bombesin- | Neuromedin B | BLAST_GENBANK |
| | | | | like peptides | [Homo sapiens] | HMMER |
| | | | | family signature: | | SPSCAN |
| | | | | R46-M56, D30-R81 | | BLIMPS_BLOCKS |
| | | | | Bombesin family | | PROFILESCAN |
| | | | | Neuromedin B | | BLAST-_PRODOM |
| | | | | Precursor | | BLAST_DOMO |
| | | | | threshold: G57 | | |
| | | | | K121 | | |
| 23 | 116 | S16 S29 S86 S93 | | | g2232301 | Motifs |
| | | | | | FMRFamide-related | BLAST_GENBANK |
| | | | | | prepropeptide | |
| | | | | | [Homo sapiens] | |
| 24 | 136 | T60 S85 S129 | N58 | Signal peptide | g6715115 | BLAST_GENBANK |
| | | S77 | | M1-A19 | agkisacutacin | Motifs |
| | | Y67 | | | | SPSCAN |
| 25 | 176 | S19 S19 S72 | | Interleukin-1: | g6694392 FIL1 | BLAST_GENBANK |
| | | S170 T6 S7 S107 | | I20-P163 | (IL1 family | Motifs |
| | | T148 | | Interleukin-1 | protein) zeta | HMMER_PFAM |
| | | | | signature: Q92- | | PROFILESCAN |
| | | | | E158 | | BLAST_DOMO |
| 26 | 134 | S102 | | Signal peptide | | Motifs |
| | | | | M1-H18 | | HMMER |

TABLE 3

| Nucleotide | Selected | Tissue Expression | Disease or Condition | |
|---|---|---|---|---|
| SEQ ID NO: | Fragments | (Fraction of Total) | (Fraction of Total) | Vector |
| 27 | 651-695 | Nervous (0.321) | Cancer (0.357) | pINCY |
| | | Cardiovascular (0.143) | Inflammation (0.250) | |
| | | Developmental (0.143) | Fetal/Cell Proliferation (0.214) | |
| 28 | 271-315 | Developmental (1.000) | Fetal/Cell Proliferation (1.000) | pINCY |
| 29 | 327-371 | Reproductive (1.000) | Cancer (0.667) | PSPORT1 |
| | | | Trauma (0.333) | |
| 30 | 640-684 | Hematopoietic/Immune (0.333) | Cancer (0.667) | pINCY |
| | | Cardiovascular (0.167) | Inflammation (0.333) | |
| | | Endocrine (0.167) | Fetal/Cell Proliferation (0.167) | |
| 31 | 1028-1072 | Cardiovascular (0.333) | Inflammation (0.667) | PSPORT1 |
| | | Nervous (0.333) | Cancer (0.333) | |
| | | Reproductive (0.333) | | |
| 32 | 271-315 | Endocrine (0.500) | Cancer (1.000) | pINCY |
| | | Reproductive (0.500) | | |
| 33 | 205-249 | Nervous (0.750) | Cancer (1.000) | PSPORT1 |
| | | Gastrointestinal (0.250) | Fetal/Cell Proliferation (0.500) | |
| 34 | 21-65 | Reproductive (0.400) | Cancer (0.400) | pINCY |
| | | Hematopoietic/Immune (0.200) | Fetal/Cell Proliferation (0.200) | |
| | | Nervous (0.200) | Inflammation (0.200) | |
| 35 | 273-317 | Nervous (1.000) | Nervous (1.000) | pINCY |
| 36 | 131-175 | Reproductive (0.333) | Cancer (0.333) | pINCY |
| | | Gastrointestinal (0.222) | Inflammation (0.222) | |
| | | Cardiovascular (0.111) | Fetal/Cell Proliferation (0.111) | |
| 37 | 58-87 | Reproductive (1.000) | Cancer (0.750) | PBLUESCRIPT |
| | 376-405 | | Inflammation (0.250) | |
| 38 | 109-168 | Reproductive (0.300) | Cancer (0.488) | pINCY |
| | 415-474 | Cardiovascular (0.143) | Inflammation (0.330) | |
| | | Nervous (0.138) | Cell Proliferation (0.172) | |
| | | Gastrointestinal (0.113) | | |
| 39 | 809-868 | Reproductive (0.625) | Cancer (0.438) | PSPORT1 |
| | 1229-1288 | Gastrointestinal (0.188) | Inflammation (0.437) | |
| | | | Cell Proliferation (0.125) | |
| 40 | 243-302 | Hematopoietic/Immune (0.727) | Inflammation (0.545) | PSPORT1 |
| | | Dermatologic (0.091) | Cell Proliferation (0.360) | |
| | | Gastrointestinal (0.091) | Cancer (0.182) | |
| | | Reproductive (0.091) | | |
| 41 | 459-518 | Nervous (0.555) | Cancer (0.500) | pINCY |
| | | Endocrine (0.111) | Neurological (0.111) | |
| | | Gastrointestinal (0.111) | | |
| | | Reproductive (0.111) | | |
| | | Cardiovascular (0.111) | | |
| 42 | 241-300 | Gastrointestinal (1.000) | Cancer (0.500) | pINCY |
| 43 | 757-801 | Reproductive (0.289) | Cancer (0.465) | |
| | | Hematopoietic/Immune (0.140) | Inflammation (0.360) | |
| | | Nervous (0.132) | Cell Proliferation (0.123) | |
| 44 | 165-209 | Hematopoietic/Immune (1.000) | Cancer (0.300) | pINCY |
| | 434-479 | | Inflammation (0.300) | |
| 45 | 1-46 | Nervous (0.375) | Cancer (0.281) | PSPORT1 |
| | | Reproductive (0.313) | Inflammation (0.313) | |
| | | Gastrointestinal (0 .093) | | |
| 46 | 866-910 | Urologic (0.500) | Cancer (0.750) | PSPORT1 |
| | | Nervous (0.250) | Inflammation (0.250) | |
| | | Reproductive (0.250) | | |
| 47 | 1029-1073 | Cardiovascular (0.234) | Cancer (0.455) | pINCY |
| | | Reproductive (0.221) | Inflammation (0.331) | |
| | | Nervous (0.182) | Cell proliferation (0.143) | |
| 48 | 76-120 | Hematopoietic/Immune (0.308) | Cancer | pINCY |
| | | Gastrointestinal (0.231) | Cell proliferation (0.143) | |
| | | Nervous (0.154) | Inflammation (0.308) | |
| 49 | 111-155 | Reproductive (0.333) | Cancer (0.750) | pINCY |
| | | Cardiovascular (0.167) | Cell proliferation (0.167) | pINCY |
| | | Developmental (0.167) | Inflammation (0.083) | |
| | | Nervous (0.167) | | |
| 50 | 218-262 | Nervous (1.000) | Cell proliferation (1.000) | PSPORT1 |
| 51 | 109-153 | Dermatologic (0.500) | pINCY | |
| | | Reproductive (0.500) | | |
| 52 | 277-321 | Nervous (1.000) | Neurological (1.000) | pINCY |

TABLE 4

| Nucleotide | | |
|---|---|---|
| SEQ ID NO: | Library | Library Description |
| 27 | BRAINOT11 | This library was constructed using RNA |
| | | isolated from brain tissue removed from |
| | | the right temporal lobe of a 5-year-old |
| | | Caucasian male during a |
| | | hemispherectomy. Pathology indicated |
| | | extensive polymicrogyria and mild to |
| | | moderate gliosis (predominantly |
| | | subpial and subcortical), consistent |
| | | with chronic seizure disorder. Family |
| | | history included a cervical neoplasm. |
| | | Family history included a cervical |
| | | neoplasm |
| 28 | PANCNOT07 | This library was constructed using RNA |
| | | isolated from the pancreatic tissue of a |
| | | Caucasian male fetus, who died at 23 |
| | | weeks' gestation. |
| 29 | PROSNON01 | This normalized prostate library was |
| | | constructed from 4.4 M independent |
| | | clones from a prostate library. Starting |
| | | RNA was made from prostate tissue |
| | | removed from a 28-year-old Caucasian |
| | | male who died from a self-inflicted |
| | | gunshot wound. The normalization and |
| | | hybridization conditions were adapted |
| | | from Soars, M.B. et al. (1994) Proc. |
| | | Natl. Acad. Sci. USA 91:9228-9232, |
| | | using a longer (19 hour) reannealing |
| | | hybridization period. |
| 30 | SPLNNOT04 | This library was constructed using RNA |
| | | isolated from the spleen tissue of a |
| | | 2-year-old Hispanic male, who died from |
| | | cerebral anoxia. |
| 31 | HEALDIT02 | This library was constructed using RNA |
| | | isolated from diseased left ventricle |
| | | tissue removed from a 56-year-old male |
| | | during a heart transplant. Patient history |
| | | included cardiovascular disease and |
| | | myocardial infarction. |
| 32 | PROSTUT12 | This library was constructed using RNA |
| | | isolated from prostate tumor tissue |
| | | removed from a 65-year-old Caucasian |
| | | male during a radical prostatectomy. |
| | | Pathology indicated an adenocarcinoma |
| | | (Gleason grade 2 + 2). Adenofibromatous |
| | | hyperplasia was also present. The patient |
| | | presented with elevated prostate specific |
| | | antigen (PSA). |
| 33 | BRAINON01 | This library was constructed and |
| | | normalized from 4.88 million independent |
| | | clones from a brain library. RNA was |
| | | made from brain tissue removed from a |
| | | 26-year-old Caucasian male during |
| | | cranioplasty and excision of a cerebral |
| | | meningeal lesion. Pathology for the |
| | | associated tumor tissue indicated a grade |
| | | 4 oligoastrocytoma in the right fronto- |
| | | parietal part of the brain. |
| 34 | BRSTTUT16 | This library was constructed using RNA |
| | | isolated from breast tumor tissue removed |
| | | from a 43-year-old Caucasian female |
| | | during a unilateral extended simple |
| | | mastectomy. Pathology indicated recurrent |
| | | grade 4, nuclear grade 3, ductal |
| | | carcinoma. Angiolymphatic space invasion |
| | | was identified. Left breast needle biopsy |
| | | indicated grade 4 ductal adenocarcinoma. |
| | | Paraffin embedded tissue was estrogen |
| | | positive. Patient history included breast |
| | | history included cervical cancer. |
| 35 | BRADDIT02 | This library was constructed using RNA |
| | | isolated from diseased choroid plexus |
| | | tissue of the lateral ventricle removed |
| | | from the brain of a 57-year-old Caucasian |
| | | male, who died from a cerebrovascular |
| | | accident. Huntington's disease, and |
| | | emphysema. |
| 36 | UTRSTMR01 | This library was constructed using 1.5 |
| | | micrograms of polyA RNA isolated from |
| | | uterine myometrial tissue removed from a |
| | | 41-year-old Caucasian female during a |
| | | vaginal hysterectomy. The myometrium |
| | | and cervix were unremarkable; the |
| | | endometrium was secretory and contained |
| | | fragments of endometrial polyps. |
| | | Pathology for associated tumor tissue |
| | | indicated uterine leiomyoma. Patient |
| | | history included ventral hernia and a |
| | | benign ovarian neoplasm. |
| 37 | TESTNOT03 | The library was constructed using RNA |
| | | isolated from testicular tissue removed |
| | | from a 37-year-old Caucasian male, who |
| | | died from liver disease. Patient history |
| | | included cirrhosis, jaundice, and liver |
| | | failure. |
| 38 | SINTFET03 | The library was constructed using RNA |
| | | isolated from small intestine tissue |
| | | removed from a Caucasian female fetus, |
| | | who died at 20 week' gestation. |
| 39 | UTRSNOT02 | The library was constructed using RNA |
| | | isolated from uterine tissue removed from |
| | | a 34-year-old Caucasian female during a |
| | | vaginal hysterectomy. Patient history |
| | | included mitral valve disorder. Family |
| | | history heart anomaly, irritable bowel |
| | | syndrome, ulcerative colotis, colon cancer, |
| | | cerebrovascular disease, type II diabetes, |
| | | and depression. |
| 40 | THYMNON04 | The normalized thymus library was |
| | | constructed using RNA isolated from |
| | | thymus tissue removed from a 3-year-old |
| | | Caucasian male, who died from anoxia. |
| 41 | TESTNOT07 | The library was constructed using RNA |
| | | isolated from testicular tissue removed |
| | | from a 31-year-old Caucasian male during |
| | | an unilateral orchiectomy (excision of |
| | | testis). Pathology indicated a mass |
| | | containing a large subcapsular hematoma |
| | | with laceration of the tunica albuginea. |
| | | The surrounding testicular parenchyma |
| | | was extensively necrotic. The patient |
| | | presented with a trunk injury. |
| 42 | LIVRTUT13 | The library was constructed using RNA |
| | | isolated from liver tumor tissue removed |
| | | from a 62-year-old Caucasian female |
| | | during partial hepatectomy and |
| | | exploratory laparotomy. Pathology |
| | | indicated metastatic intermediate grade |
| | | neuroendocrine carcinoma, consistent with |
| | | islet cell tumor, forming nodules ranging |
| | | in size, in the lateral and medial left |
| | | liver lobe. The pancreas showed fibrossis, |
| | | chronic inflammation and fat necrosis |
| | | consistent with pseudocyst. The |
| | | gallbladder showed mild chronic |
| | | cholecystitis. Patient history included |
| | | maligant neoplasm of the pancreas tail, |
| | | pulmonary embolism, hyperlipidemia |
| | | thrombophlebitis, joint pain multiple |
| | | joints, type II diabetes, benign |
| | | hypertension, and cerebrovascular disease. |
| | | Family history included pancreas cancer, |
| | | secondary liver cancer, benign |
| | | hypertension, and hyperlipidemia. |
| 43 | LUNGFET03 | Library was constructed using RNA |
| | | isolated from lung tissue removed from a |
| | | Caucasian female fetus, who died at 20 |
| | | weeks' gestation. |
| 44 | TLYHNOT08 | Library was constructed using RNA |
| | | isolated from anergicallogenic |
| | | T-lymphocyte tissue removed from an |
| | | adult (40-50-year-old) Caucasian male. |
| | | The cells were incubated for 3 days in |
| | | the presence of OKT3 mAb |
| | | (1 microgram/mlOKT3 and 5% human |
| | | serum. |
| 45 | UTRSNOT02 | Library was constructed using RNA |
| | | isolated from uterine tissued removed |
| | | from a 34-year-old Caucasian female |
| | | during a vaginal hysterectomy. Patient |
| | | history included mitral valve disorder. |
| | | Family history included stomach cancer, |
| | | congential heart anomaly, irritable |
| | | bowel syndrome, ulcerative colitis, colon |
| | | cancer, cerebrovascular disease, type II |
| | | diabetes, and depression. |
| 46 | PROSTUT05 | Library was constructed using RNA |
| | | isolated prostate tumor tissue removed |
| | | from a 69-year-old Caucasian male during |
| | | a radical prostatectomy. Pathology |
| | | indicated adenocarcinoma (Gleason grade |
| | | 3 + 4). Adenofibromatous hyperplasia |
| | | was also present. Family history included |
| | | congestive heart failure, multiple myeloma |
| | | hyperlipidemia, and rheumatoid arthritis. |
| 47 | LUNGNOT12 | Library was constructed using RNA |
| | | isolatedfrom lung tissue remove form |
| | | a 78-year-old Caucasian male during a |
| | | segmental lung resection and regional |
| | | lymph resection. Pathology indicated |
| | | fibrosis pleura was puckered, but not |
| | | invaded Pathology for the associated |
| | | tumor tissue indicated an invasive |
| | | pulmonary grade 3 adenocarcinoma. |
| | | Patient history included cerebrovasular |
| | | disease, arteriosclerotic coronary |
| | | artery disease, thrombophlebitis, chronic |
| | | obstructive pulmonary disease, and |
| | | asthma. Family history included |
| | | intracranial hematoma, cerebrovascular |
| | | disease, arteriosclerotic coronary artery |
| | | disease, and type I diabetes. |
| 48 | THYRNOT03 | Library was constructed using RNA |
| | | isolated from thyroid tissue removed |
| | | from the left thyroid of a 28-year-old |
| | | Caucasian female during a complete |
| | | thyroidectomy. Pathology indicated a |
| | | small nodule of adenomatous hyperplasia |
| | | present in the left thyroid. Pathology for |
| | | the associated tumor tissue indicated |
| | | dominant follicular adenoma, forming a |
| | | well-encapsulated mass in the left thyroid. |
| 49 | PROSTUT08 | Library was constructed using RNA |
| | | isolated from prostate tumor tissue |
| | | removed from a 60-year-old Caucasian |
| | | male during radical prostatectomy and |
| | | regional lymph node excision. Pathology |
| | | indicated an adenocarcinoma |
| | | (Gleason grade 3 + 4). |
| | | Adenofibromatous hyperplasia was also |
| | | present. Patient history included a kidney |
| | | cyst, and hematuria. Family history |
| | | included tuberculosis, cerebrovasular |
| | | disease, and arteriosclerotic coronary |
| | | artery disease. |
| 50 | HNT2AZS07 | This subtracted library was constructed |
| | | from RNA isolated from a hNT2 cell line |
| | | (derived from a human teratocarcinoma |
| | | that exhibited properties characteristic |
| | | of a committed neuronal precursor) |
| | | treated for three days with 0.35 |
| | | micromolar AZ. The hybridization probe |
| | | for subtraction was derived from a |
| | | similarly constructed library from |
| | | untreated hNT2 cells. 3.08 M clones |
| | | from the AZ-treated library were subjected |
| | | to three rounds of subtractive |
| | | hybridization with 3.04 M clones from the |
| | | untreated library. Subtractive hybridization |
| | | conditions wre based on the |
| | | methodologies of Swaroop et al. |
| | | (NAR (1991) 19:1954) and Bonaldo et al. |
| | | (Genome Research (1996) 6:791). |
| 51 | SKIRNOT01 | Library was constructed using RNA |
| | | isolated from skin tissue removed from the |
| | | breast of a 26-year-old Caucasian female |
| | | during bilateral reduction mammopplasty. |
| 52 | BRABDIR01 | Library was constructed using RNA |
| | | isolated from diseased cerebellum tissue |
| | | removed from the brain of a 57-year-old |
| | | Caucasian male, who died from a |
| | | cerebrovascular accident. |

TABLE 5

| | | | Parameter |
|---|---|---|---|
| Program | Description | Reference | Threshold |
| ABI | program that removes vector sequences and | Applied Biosystems, Foster City, CA. | |
| FACTURAA | masks ambiguous bases in nucleic acid sequences. | | |
| ABI/PARACEL | A Fast Data Finder useful in comparing and | Applied Biosystems, Foster City, CA; | Mismatch |
| FDF | annotating amino acid or nucleic acid sequences. | Paracel Inc., Pasadena, CA. | <50% |
| ABI | A program that assembles nucleic acid sequences. | Applied Biosystems, Foster City, CA. | |
| AutoAssembler | | | |
| BLAST | A Basic Local Alignment Search Tool useful in | Altschul, S. F. et al. (1990) J. Mol. Biol. | ESTs: |
| | sequence similarity search for amino acid and | 215:403-410; Altschul, S. F. et al. (1997) | Probability |
| | nucleic acid sequences. BLAST includes five | Nucleic Acids Res. 25:3389-3402. | value = 1.08E−8 |
| | functions: blastp, blastn, blastx, tblastn, and tblastx. | | or less |
| | | | Full length |
| | | | sequences: |
| | | | Probability |
| | | | value =1.0E−10 |
| | | | or less |
| FASTA | A Pearson and Lipman algorithm that searches for | Pearson, W. R. and D. J. Lipman (1988) Proc. | ESTs: fasta E |
| | similarity between a query sequence and a group of | Natl. Acad Sci. USA 85:2444-2448; Pearson, | value = 1.06E−6 |
| | sequences of the same type. FASTA comprises as | W. R. (1990) Methods Enzymol. 183:63-98; | Assembled ESTs: |
| | least five functions: fasta, tfasta, fastx, tfastx, and | and Smith, T. F. and M. S. Waterman (1981) | fasta Identity = |
| | ssearch. | Adv. Appl. Math. 2:482-489. | 95% or greater |
| | | | and Match |
| | | | length = 200 |
| | | | bases or greater; |
| | | | fastx E |
| | | | value = 1.0E−8 |
| | | | or less |
| | | | Full Length |
| | | | sequences: |
| | | | fastx score = 100 |
| | | | or greater |
| BLIMPS | A BLocks IMProved Searcher that matches a | Henikoff, S. and J. G. Henikoff (1991) Nuleic | Probability |
| | sequence against those in BLOCKS, PRINTS, | Acids Res. 19:6565-6572; Henikoff, J. G. and | value = 1.0E−3 |
| | DOMO, PRODOM, and PFAM databases to search | S. Henikoff (1996) Methods Enzymol. | or less |
| | for gene families, sequence homology, and structural | 266:88-105; and Attwood, T. K. et al. (1997) | |
| | fingerprint regions. | J. Chem. Inf. Comput. Sci 37:417-424. | |
| HMMER | An algorithm for searching a query sequence against | Krogh, A. et al. (1994) J. Mol. Biol. | PFAM hits: |
| | hidden Markov model (HIVIM)-based databases of | 235:1501-1531; Sonnhammer, E. L. L. et al. | Probability |
| | protein family consensus sequences, such as PFAM. | (1988) Nucleic Acids Res. 26:320-322; | value = 1.0E−3 |
| | | Durbin, R. et al. (1998) Our World View, in a | or less |
| | | Nutshell, Cambridge Univ. Press, pp. 1-350. | Signal peptide |
| | | | hits: Score = 0 |
| | | | or greater |
| ProfileScan | An algorithm that searches for structural and sequence | Gribskov, M. et al. (1988) CABIOS 4:61-66; | Normalized |
| | motifs in protein sequences that match sequence | Gribskov, M. et al. (1988) Methos Enzymol. | quality |
| | patterns defined in Prosite. | 183:146-159; Bairoch, A. et al. (1997) | score ≧ GCG- |
| | | Nucleic Acids Res. 25:217-221. | specified “HIGH” |
| | | | value for that |
| | | | particular |
| | | | Prosite motif. |
| | | | Generally, |
| score = 1.4-2.1. | | | |
| Phred | A base-calling algorithm that examines automated | Ewing, B. et al. (1998) Genome Res. | |
| | sequencer traces with high sensitivity and probability. | 8:175-185; Ewing, B. and P. Green | |
| | | (1998) Genome Res. 8:186-194 | |
| Phrap | A Phils Revised Assembly Program including SWAT | Smith, T. F. and M. S. Waterman (1981) Adv. | Score = 120 or |
| | and CrossMatch, programs based on efficient | Appl. Math. 2:482-489; Smith, T. F. and M. S. | greater; |
| | implementation of the Smith-Waterman algorithm, | Waterman (1981) J. Mol. Biol. 147:195-197; | Match |
| | useful in searching sequence homology and assembling | and Green, P., University of Washington, | length = 56 or |
| | DNA sequences. | Seattle, WA. | greater |
| Consed | A graphical tool for viewing and editing Phrap | Gordon, D. et al. (1998) Genome Res. 8:195-202. | |
| | assemblies. | | |
| SPScan | A weight matrix analysis program that scans protein | Nielson, H. et al. (1997) Protein Engineering | Score = 3.5 or |
| | sequences for the presence of secretory signal peptides. | 10:1-6; Claverie, J. M. and S. Audic (1997) | greater |
| | | CABIOS 12:431-439. | |
| TMAP | A program that uses weight matrices to delineate | Persson, B. and P. Argos (1994) J. Mol. Biol | |
| | transmembrane segments on protein sequences and | 237:182-192; Persson, B. and P. Argos (1996) | |
| | determine orientation. | Protein Sci. 5:363-371. | |
| TMHMMER | A program that uses a hidden Markov model (HMM) | Sonnhammer, E. L. et al. (1998) proc. Sixth Intl. | |
| | to delineate transmembrane segments on protein | Conf. on Intelligent Systems for Mol. Biol., | |
| | sequences and determine orientation. | Glasgow et al., eds., The Am. Assoc. for Artificial | |
| | | Intelligence Press, Menlo Park, CA, pp. 175-182. | |
| Motifs | A program that searches amino acid sequences for | Bairoch, A. et al. (1997) Nuleic Acids Res. | |
| | patterns that matched those defined in Prosite. | 25:217-221; | |
| | | Wisconsin Package Program Manual, version 9, page | |
| | | M51-59, Genetics Computer Group, Madison, WI. | |

What is claimed is:

1. An isolated polypeptide selected from the group consisting of:
a) a polypeptide comprising the amino acid sequence of SEQ ID NO: 16,
b) a polypeptide comprising a naturally occurring amino acid sequence at least 90% identical to the amino acid sequence of SEQ ID NO:16,
c) a biologically active fragment of a polypeptide having the amino acid sequence of SEQ ID NO:16, and
d) an immunogenic fragment of a polypeptide having the amino acid sequence of SEQ ID NO:16.

2. An isolated polypeptide of claim 1 selected from the group consisting of SEQ ID NO:1-26.

3. An isolated polynucleotide encoding a polypeptide of claim 1.

4. An isolated polynucleotide encoding a polypeptide of claim 2.

5. An isolated polynucleotide of claim 4 selected from the group consisting of SEQ ID NO:27-52.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide has an amino acid sequence selected from the group consisting of SEQ ID NO:1-26.

11. An isolated antibody which specifically binds to a polypeptide selected from the group consisting of:
a) a polypeptide comprising the amino acid sequence of SEQ ID NO: 16,
b) a polypeptide comprising a naturally occurring amino acid sequence at least 90% identical to the amino acid sequence of SEQ ID NO:16,
c) a biologically active fragment of a polypeptide having the amino acid sequence of SEQ ID NO:16, and
d) an immunogenic fragment of a polypeptide having the amino acid sequence of SEQ ID NO:16.

12. An isolated polynucleotide selected from the group consisting of:
a) a polynucleotide comprising the polynucleotide of SEQ ID NO:42,
b) a polynucleotide comprising a naturally occurring polynucleotide sequence at least 90% identical to the polynucleotide sequence of SEQ ID NO:42,
c) a polynucleotide complementary to a polynucleotide of a),
d) a polynucleotide complementary to a polynucleotide of b), and
e) an RNA equivalent of a)-d).

13. An isolated polynucleotide comprising at least 60 contiguous nucleotides of a polynucleotide of claim 12.

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. A method of claim 14, wherein the probe comprises at least 60 contiguous nucleotides.

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide has an amino acid sequence selected from the group consisting of SEQ ID NO:1-26.

19. A method for treating a disease or condition associated with decreased expression of functional EXCS, comprising administering to a patient in need of such treatment the composition of claim 17.

20. A method of screening a compound for effectiveness as an agonist of a polypeptide of claim 1, the method comprising:
a) exposing a sample comprising a polypeptide of claim I to a compound, and
b) detecting agonist activity in the sample.

21. A composition comprising an agonist compound identified by a method of claim 20 and a pharmaceutically acceptable excipient.

22. A method for treating a disease or condition associated with decreased expression of functional EXCS, comprising administering to a patient in need of such treatment a composition of claim 21.

23. A method of screening a compound for effectiveness as an antagonist of a polypeptide of claim 1, the method comprising:
a) exposing a sample comprising a polypeptide of claim I to a compound, and
b) detecting antagonist activity in the sample.

24. A composition comprising an antagonist compound identified by a method of claim 23 and a pharmaceutically acceptable excipient.

25. A method for treating a disease or condition associated with overexpression of functional EXCS, comprising administering to a patient in need of such treatment a composition of claim 24.

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. A method of screening for a compound that modulates the activity of the polypeptide of claim 1, the method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30. A diagnostic test for a condition or disease associated with the expression of EXCS in a biological sample, the method comprising:
a) combining the biological sample with an antibody of claim 11, under conditions suitable for the antibody to bind the polypeptide and form an antibody:polypeptide complex, and
b) detecting the complex, wherein the presence of the complex correlates with the presence of the polypeptide in the biological sample.

31. The antibody of claim 11, wherein the antibody is:
a) a chimeric antibody,
b) a single chain antibody,
c) a Fab fragment,
d) a F(ab′)₂ fragment, or
e) a humanized antibody.

32. A composition comprising an antibody of claim 11 and an acceptable excipient.

33. A method of diagnosing a condition or disease associated with the expression of EXCS in a subject, comprising administering to said subject an effective amount of the composition of claim 32.

34. A composition of claim 32, wherein the antibody is labeled.

35. A method of diagnosing a condition or disease associated with the expression of EXCS in a subject, comprising administering to said subject an effective amount of the composition of claim 34.

36. A method of preparing a polyclonal antibody with the specificity of the antibody of claim 11, the method comprising:
a) immunizing an animal with a polypeptide having the amino acid sequence of SEQ ID NO: 16, or an immunogenic fragment thereof, under conditions to elicit an antibody response,
b) isolating antibodies from said animal, and
c) screening the isolated antibodies with the polypeptide, thereby identifying a polyclonal antibody which binds specifically to a polypeptide having the amino acid sequence of SEQ ID NO:16.

37. A polyclonal antibody produced by a method of claim 36.

38. A composition comprising the polyclonal antibody of claim 37 and a suitable carrier.

39. A method of making a monoclonal antibody with the specificity of the antibody of claim 11, the method comprising:
a) immunizing an animal with a polypeptide having the amino acid sequence of SEQ ID NO:16, or an immunogenic fragment thereof, under conditions to elicit an antibody response,
b) isolating antibody producing cells from the animal,
c) fusing the antibody producing cells with immortalized cells to form monoclonal antibody-producing hybridoma cells,
d) culturing the hybridoma cells, and
e) isolating from the culture monoclonal antibody which binds specifically to a polypeptide having the amino acid sequence of SEQ ID NO: 16.

40. A monoclonal antibody produced by a method of claim 39.

41. A composition comprising the monoclonal antibody of claim 40 and a suitable carrier.

42. The antibody of claim 11, wherein the antibody is produced by screening a Fab expression library.

43. The antibody of claim 11, wherein the antibody is produced by screening a recombinant immunoglobulin library.

44. A method of detecting a polypeptide having the amino acid sequence of SEQ ID NO: 16 in a sample, the method comprising:
a) incubating the antibody of claim 11 with a sample under conditions to allow specific binding of the antibody and the polypeptide, and
b) detecting specific binding, wherein specific binding indicates the presence of a polypeptide having the amino acid sequence of SEQ ID NO:16 in the sample.

45. A method of purifying a polypeptide having the amino acid sequence of SEQ ID NO:16 from a sample, the method comprising:
a) incubating the antibody of claim 11 with a sample under conditions to allow specific binding of the antibody and the polypeptide, and
b) separating the antibody from the sample and obtaining the purified polypeptide having the amino acid sequence of SEQ ID NO: 16.

46. A microarray wherein at least one element of the microarray is a polynucleotide of claim 13.

47. A method of generating a transcript image of a sample which contains polynucleotides, the method comprising:
a) labeling the polynucleotides of the sample,
b) contacting the elements of the microarray of claim 46 with the labeled polynucleotides of the sample under conditions suitable for the formation of a hybridization complex, and
c) quantifying the expression of the polynucleotides in the sample.

48. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, and wherein said target polynucleotide is a polynucleotide of claim 12.

49. An array of claim 48, wherein said first oligonucleotide or polynucleotide sequence is completely complementary to at least 30 contiguous nucleotides of said target polynucleotide.

50. An array of claim 48, wherein said first oligonucleotide or polynucleotide sequence is completely complementary to at least 60 contiguous nucleotides of said target polynucleotide.

51. An array of claim 48, wherein said first oligonucleotide or polynucleotide sequence is completely complementary to said target polynucleotide.

52. An array of claim 48, which is a microarray.

53. An array of claim 48, further comprising said target polynucleotide hybridized to a nucleotide molecule comprising said first oligonucleotide or polynucleotide sequence.

54. An array of claim 48, wherein a linker joins at least one of said nucleotide molecules to said solid substrate.

55. An array of claim 48, wherein each distinct physical location on the substrate contains multiple nucleotide molecules, and the multiple nucleotide molecules at any single distinct physical location have the same sequence, and each distinct physical location on the substrate contains nucleotide molecules having a sequence which differs from the sequence of nucleotide molecules at another distinct physical location on the substrate.

56. A polypeptide of claim 1, comprising the amino acid sequence of SEQ ID NO:1.

57. A polypeptide of claim 1, comprising the amino acid sequence of SEQ ID NO:2.

58. A polypeptide of claim 1, comprising the amino acid sequence of SEQ ID NO:3.

59. A polypeptide of claim 1, comprising the amino acid sequence of SEQ ID NO:4.

60. A polypeptide of claim 1, comprising the amino acid sequence of SEQ ID NO:5.

61. A polypeptide of claim 1, comprising the amino acid sequence of SEQ ID NO:6.

62. A polypeptide of claim 1, comprising the amino acid sequence of SEQ ID NO:7.

63. A polypeptide of claim 1, comprising the amino acid sequence of SEQ ID NO:8.

64. A polypeptide of claim 1, comprising the amino acid sequence of SEQ ID NO:9.

65. A polypeptide of claim 1, comprising the amino acid sequence of SEQ ID NO:10.

66. A polypeptide of claim 1, comprising the amino acid sequence of SEQ ID NO:11.

67. A polypeptide of claim 1, comprising the amino acid sequence of SEQ ID NO:12.

68. A polypeptide of claim 1, comprising the amino acid sequence of SEQ ID NO:13.

69. A polypeptide of claim 1, comprising the amino acid sequence of SEQ ID NO:14.

70. A polypeptide of claim 1, comprising the amino acid sequence of SEQ ID NO:15.

71. A polypeptide of claim 1, comprising the amino acid sequence of SEQ ID NO:16.

72. A polypeptide of claim 1, comprising the amino acid sequence of SEQ ID NO:17.

73. A polypeptide of claim 1, comprising the amino acid sequence of SEQ ID NO:18.

74. A polypeptide of claim 1, comprising the amino acid sequence of SEQ ID NO:19.

75. A polypeptide of claim 1, comprising the amino acid sequence of SEQ ID NO:20.

76. A polypeptide of claim 1, comprising the amino acid sequence of SEQ ID NO:21.

77. A polypeptide of claim 1, comprising the amino acid sequence of SEQ ID NO:22.

78. A polypeptide of claim 1, comprising the amino acid sequence of SEQ ID NO:23.

79. A polypeptide of claim 1, comprising the amino acid sequence of SEQ ID NO:24.

80. A polypeptide of claim 1, comprising the amino acid sequence of SEQ ID NO:25.

81. A polypeptide of claim 1, comprising the amino acid sequence of SEQ ID NO:26.

82. A polynucleotide of claim 12, comprising the polynucleotide sequence of SEQ ID NO:27.

83. A polynucleotide of claim 12, comprising the polynucleotide sequence of SEQ ID NO:28.

84. A polynucleotide of claim 12, comprising the polynucleotide sequence of SEQ ID NO:29.

85. A polynucleotide of claim 12, comprising the polynucleotide sequence of SEQ ID NO:30.

86. A polynucleotide of claim 12, comprising the polynucleotide sequence of SEQ ID NO:31.

87. A polynucleotide of claim 12, comprising the polynucleotide sequence of SEQ ID NO:32.

88. A polynucleotide of claim 12, comprising the polynucleotide sequence of SEQ ID NO:33.

89. A polynucleotide of claim 12, comprising the polynucleotide sequence of SEQ ID NO:34.

90. A polynucleotide of claim 12, comprising the polynucleotide sequence of SEQ ID NO:35.

91. A polynucleotide of claim 12, comprising the polynucleotide sequence of SEQ ID NO:36.

92. A polynucleotide of claim 12, comprising the polynucleotide sequence of SEQ ID NO:37.

93. A polynucleotide of claim 12, comprising the polynucleotide sequence of SEQ ID NO:38.

94. A polynucleotide of claim 12, comprising the polynucleotide sequence of SEQ ID NO:39.

95. A polynucleotide of claim 12, comprising the polynucleotide sequence of SEQ ID NO:40.

96. A polynucleotide of claim 12, comprising the polynucleotide sequence of SEQ ID NO:41.

97. A polynucleotide of claim 12, comprising the polynucleotide sequence of SEQ ID NO:42.

98. A polynucleotide of claim 12, comprising the polynucleotide sequence of SEQ ID NO:43.

99. A polynucleotide of claim 12, comprising the polynucleotide sequence of SEQ ID NO:44.

100. A polynucleotide of claim 12, comprising the polynucleotide sequence of SEQ ID NO:45.

101. A polynucleotide of claim 12, comprising the polynucleotide sequence of SEQ ID NO:46.

102. A polynucleotide of claim 12, comprising the polynucleotide sequence of SEQ ID NO:47.

103. A polynucleotide of claim 12, comprising the polynucleotide sequence of SEQ ID NO:48.

104. A polynucleotide of claim 12, comprising the polynucleotide sequence of SEQ ID NO:49.

105. A polynucleotide of claim 12, comprising the polynucleotide sequence of SEQ ID NO:50.

106. A polynucleotide of claim 12, comprising the polynucleotide sequence of SEQ ID NO:51.

107. A polynucleotide of claim 12, comprising the polynucleotide sequence of SEQ ID NO:52.
